(19) United States
(12) Patent Application Publication
(10) Pub. No.: US 2013/0273088 A1
(43) Pub. Date: Oct. 17, 2013

(54) EPITOPES OF HERPES SIMPLEX VIRUS

(71) Applicants: Anthony Lawrence Cunningham, McMahons Point (AU); Min Kim, Castle Hill (AU)

(72) Inventors: Anthony Lawrence Cunningham, McMahons Point (AU); Min Kim, Castle Hill (AU)

(21) Appl. No.: 12/667,923

(22) PCT Filed: Jul. 7, 2008
(86) PCT No.: PCT/AU2008/000996

§ 371 Date: Jan. 6, 2010

(30) Foreign Application Priority Data
Jul. 6, 2007 (AU) 2007903674

(51) Int. Cl. C07K 14/035

(52) U.S. Cl. CPC C07K 14/035
USPC 424/186.1; 435/39; 530/326; 530/395; 536/23.72

(57) ABSTRACT

The present invention relates to diagnosis, prevention and treatment of Herpes simplex viruses and infection. In particular embodiments the present invention relates to methods and compositions for the prophylactic or therapeutic immunization against of infections of HSV. The present invention also relates to methods and compositions for diagnosis of the presence of and level of immunity to HSV. The invention also relates to peptide epitopes of HSV, in particular peptide epitopes of HSV2 glycoprotein D, to compositions thereof and to the use of such epitopes and compositions in methods for diagnosis, prevention and treatment of HSV.

### CROSS REFERENCE TO RELATED APPLICATION

This application claims the benefit of Australian Provisional patent Application No. 2007903674 filed 6 Jul. 2007, which is incorporated herein by reference in its entirety.

### FIELD OF THE INVENTION

The present invention relates to diagnosis, prevention and treatment of Herpes simplex viruses and infection. In particular embodiments the present invention relates to methods and compositions for the prophylactic or therapeutic immunization against of infections of HSV. The present invention also relates to methods and compositions for diagnosis of the presence of and level of immunity to HSV. The invention also relates to peptide epitopes of HSV, in particular peptide epitopes of HSV2 glycoprotein D, to compositions thereof and to the use of such epitopes and compositions in methods for diagnosis, prevention and treatment of HSV.

### BACKGROUND

Human herpes simplex virus (HSV) is a member of the herpesviridae family of viruses whose genomes consist of a single large double-stranded DNA molecule. HSV-1 and HSV-2 (HHV-1 and HHV-2) are closely related. HSV-1 causes predominantly oral, but also genital herpes. HSV-2 is responsible for genital herpes but rarely also may cause the oral form. 80% of the general population is infected with HSV-1 and 22% with HSV-2. The prevalence is much higher in some developing countries, for example the HSV-2 infection rate is up to 50% in some African countries.

HSV also may cause other primary and recurrent infections of mucous membranes, such as gingivostomatitis and keratoconjunctivitis. Neonatal HSV infection and HSV infections of immuno-comprised individuals (encephalitis, visceral HSV, Kaposi varicella-like eruption) are highly dangerous and associated with high morbidity.

There are drugs available for controlling acute HSV outbreaks, such as aciclovir valaciclovir, famiclovir and penciclovir. After initial infection, all herperviridae persist permanently in the infected individual, preferentially in neuronal cells and become dormant (latency). Upon certain challenges to the immune system, such as stress, high UV radiation, immuno-impairment or deficiency, the viruses are induced and replicate again, causing a new outbreak. The human immune-system generates antibodies and cytotoxic T-cells against the virus, but is not able to eradicate it from the body and cannot prevent further outbreaks. Current therapies also do not eradicate HSV. Outbreaks will occur regularly but with varying frequency and severity.

In humans and/or murine models HSV specific CD4 and CD8 T-lymphocytes play a central role in controlling primary and recurrent HSV infections; in recovery from infection and in restricting HSV spread in the nervous system. They are recruited to sites of productive HSV infection or reactivation in the DRG and skin. In skin the immunoreactive cells responsible for controlling the transmitted HSV include the normal constituents of the squamous epidermis, keratinocytes and Langerhans cells, and infiltrating cells: first predominantly monocyte/macrophages and CD4 lymphocytes and later predominantly CD8 lymphocytes, as shown by immunohistochemistry and direct T-cell cloning from lesions. Infection of epidermal keratinocytes induces the secretion of a sequence of chemokines and cytokines which is reflected in the whole lesion in vivo i.e. firstly IFN-α and β chemokines and then interleukin (IL)-12 followed by IL-1 and IL-6 (Mikloska et al., 1998, In vivo production of cytokines and beta (C-C) chemokines in human recurrent herpes simplex lesions—do herpes simplex virus-infected keratinocytes contribute to their production? J Infect Dis 177: 287). The β chemokines may assist in chemotaxis of monocytes, CD4 and CD8 lymphocytes into lesions. IL-12 may entrain Th1 patterns of cytokine response from HSV antigen stimulated CD4 (and CD8) lymphocytes, especially IFN-γ. IFN-α and -γ synergise to inhibit infection of keratinocytes after transmission from axon termini.

HSV1 or 2 down-regulate MHC class I expression by epidermal keratinocytes and this is reversed by IFN-γ mainly secreted by CD4 lymphocytes infiltrating the lesion. The CD8 lymphocytes will obviously not recognise the infected keratinocytes until MHC I is restored on their surface by IFN-γ secreted by CD4 lymphocytes. Both CD4 and CD8 cytotoxic T lymphocytes (CTLs) have been isolated from genital lesions ex vivo and shown to have cytotoxic activity (Koelle et al., 1998, Recognition of herpes simplex virus type 2 tegument proteins by CD4 T cells infiltrating human genital herpes lesion, J Virol 72:7476). The CD8 lymphocyte infiltrate appears to correlate with virus eradication from the skin. CD4 CTLs were also shown to recognise HSV2 tegument proteins especially VP16 and VP22 (Koelle et al., 1998, Recognition of herpes simplex virus type 2 tegument proteins by CD4 T cells infiltrating human genital herpes lesion, J Virol 72:7476). These CD4 CTL probably act early, and CD8 CTL late in controlling HSV.

Previous work from the laboratory of the present inventors has shown that both human CD4 and CD8 T-lymphocytes recognise IFN-γ stimulated HSV1 infected keratinocytes. Using vaccinia virus recombinants expressing HSV2 proteins and blood CD4 lymphocytes restimulated in vitro the present inventors have shown that CD8 T-lymphocytes recognised immediate early (1E)/early (E) proteins, whereas CD4 T-lymphocytes recognised late HSV1 or HSV2 structural proteins, especially gD2 (Mikloska and Cunningham, 1998, Herpes simplex virus type I glycoproteins gD, gC and gD are major targets for CD4 T-lymphocyte cytotoxicity in HLA-DR expressing human epidermal keratinocytes, J Gen Virol 79 (Pt 2):353) complementing earlier studies demonstrating gD can stimulate human CD4 helper cells. Parallel studies in mice have also showed CD4 lymphocyte specificity for gD.

Successful trials of gD2 immunization in mice and guinea pigs preceded human trials of immunization with recombinant gD2 vaccine mixed with the adjuvants alum and deacylated monophosphoryl lipid A (dMPL). The latter were shown to substantially induce protection (>70%) (Stanberry et al., 2002, Glycoprotein-D-adjuvant vaccine to prevent genital herpes, N Engl J Med 347:1652) against genital herpes disease in HSV1 and 2 seronegative but not in HSV1 seropositive women. Prior natural HSV1 infection reduced development of HSV2 genital herpes disease. gD2 has also been shown to induce interferon gamma secretion from the PBMCs of similarly immunized patients when stimulated in vitro.

Type-specific serological tests are the most commonly used diagnostic tools available on the market and are capable of detecting antibodies that develop in the first several weeks of infection and persist indefinitely. Most detect glycoprotein G-specific IgG and have varying sensitivities, especially soon after infection. There are currently no convenient tests for T-cell immunity against HSV1 or HSV2.

There thus remains a need for improved agents for use in treatment, prevention and diagnosis of HSV infection.

### SUMMARY OF THE INVENTION

As described herein the present inventors have identified immunodominant peptides of glycoprotein D of HSV2 recognised by bulk human CD4 lymphocytes in the majority of HSV2 seropositive subjects by screening a gD2 peptide library. The present application also describes their MHC II restriction and whether such peptides were also recognised by HSV1+ subjects.

The results presented herein provide substantial advantages over previous attempts to identify potential diagnostic or therapeutic agents for HSV which were, for example, limited to those defining a single peptide or a preliminary scan of gD 1 with large peptides using older insensitive T cell proliferation assays, defining relatively few epitopes. MHC H restriction or HSV1/2 cross reactivity was not examined (Damhof et al., 1993, T cell responses to synthetic peptides of herpes simplex virus type 1 glycoprotein D in naturally infected individuals, Arch Virol 130:187; DeFreitas et al., 1985, Human T-lymphocyte response in vitro to synthetic peptides of herpes simplex virus glycoprotein D, Proc Natl Acad Sci USA 82: 3425)

Such studies provide an empirical basis for cross reactive and cross protective epitopes between gD of HSV1 and HSV2 suspected from the vaccine studies. A vaccine effective against both genital HSV1 and 2 infection and disease would be advantageous in view of the recent increasing incidence of genital HSV1 disease especially in adolescence.

In a first aspect of the invention there is provided an isolated immunogenic Herpes simplex virus (HSV) glycoprotein D peptide comprising an amino acid sequence selected from the group consisting of SEQ ID Nos 1 to 39 or an immunogenic fragment or variant thereof.

In one embodiment, the peptide comprises an amino acid sequence selected from the group consisting of SEQ ID NO: 2, SEQ ID NO: 4, SEQ ID NO: 10, SEQ ID NO: 12, SEQ ID NO: 24, SEQ ID NO: 26, SEQ ID NO: 30, SEQ ID NO: 34 and SEQ ID NO: 36.

In one embodiment, the peptide comprises an amino acid sequence selected from the group consisting of SEQ ID NO:1, SEQ ID NO: 3, SEQ ID NO: 5, SEQ ID NO: 9, SEQ ID NO: 11, SEQ ID NO: 13; SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one embodiment, the peptide consists of an amino acid sequence selected from the group consisting of SEQ ID NO: 2, SEQ ID NO: 4, SEQ ID NO: 10, SEQ ID NO: 12, SEQ ID NO: 24, SEQ ID NO: 26, SEQ ID NO: 30, SEQ ID NO: 34 and SEQ ID NO: 36.

In one embodiment, the peptide consists of an amino acid sequence selected from the group consisting of SEQ ID NO:1, SEQ ID NO: 3, SEQ ID NO: 5, SEQ ID NO: 9, SEQ ID NO: 11, SEQ ID NO: 13, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one embodiment the peptide consists of an amino acid sequence selected from SEQ ID Nos 1 to 39. In one embodiment the peptide consists of an amino acid sequence selected from SEQ ID Nos 41 to 76 or an immunogenic fragment or variant thereof.

In one embodiment, the peptide has binding specificity for at least one class II major histocompatability complex protein. In one embodiment the peptide has binding specificity for either or both of:

(a) an HLA-DR protein

(b) an HLA-DQ protein.

In one embodiment the HLA-DR protein is selected from the group consisting of HLA DRB1*0101, HLA DRB1*0301, HLA DRB1*0401, HLA DRB1*0404, HLA DRB1*0405, HLA DRB1*0701, HLA DRB1*1101, HLA DRB1*1302, HLA DRB1*1501 and HLA DRB3*0101.

In one embodiment the peptide further comprises a component portion of a fusion protein or polypeptide. In one embodiment the fusion protein comprises a plurality is of isolated immunogenic HSV glycoprotein D peptides. In one embodiment the fusion protein or polypeptide comprises a polypeptide sequence unrelated to the immunogenic HSV glycoprotein D peptides. In one embodiment the HSV glycoprotein D peptide is an HSV glycoprotein D2 peptide.

In a second aspect of the invention there is provided a polynucleotide sequence comprising a nucleic acid sequence encoding a peptide of the first aspect. In one embodiment the polynucleotide sequence comprises a nucleic acid sequence encoding a fusion protein or polypeptide comprising one or a plurality of peptide(s) of the first aspect. In one embodiment the polynucleotide sequence is provided in a vector. In one embodiment, the polynucleotide sequence or vector is provided in a host cell.

In a third aspect of the invention there is provided a pharmaceutical composition comprising at least one immunogenic HSV glycoprotein D peptide, said peptide comprising an amino acid sequence selected from the group consisting of SEQ ID Nos 1 to 39 or an immunogenic fragment or variant thereof, together with a pharmaceutically acceptable carrier, adjuvant or excipient.

In one embodiment the composition comprises a peptide comprising an amino acid sequence selected from the group consisting of SEQ ID NO:1, SEQ ID NO: 2, SEQ ID NO: 3, SEQ ID NO: 4, SEQ ID NO: 5, SEQ ID NO: 9, SEQ ID NO: 10, SEQ ID NO: 11, SEQ ID NO: 12, SEQ ID NO: 13, SEQ ID NO: 23, SEQ ID NO: 24, SEQ ID NO: 25, SEQ ID NO: 26, SEQ ID NO: 27, of SEQ ID NO: 29, SEQ ID NO: 30, SEQ ID NO: 31, SEQ ID NO: 33, SEQ ID NO: 34 SEQ ID NO: 35, SEQ ID NO: 36 and SEQ ID NO: 37.

In one embodiment the composition comprises a peptide consisting of an amino acid sequence selected from the group consisting of SEQ ID NO:1, SEQ ID NO: 2, SEQ ID NO: 3, SEQ ID NO: 4, SEQ ID NO: 5, SEQ ID NO: 9, SEQ ID NO: 10, SEQ ID NO: 11, SEQ ID NO: 12, SEQ ID NO: 13, SEQ ID NO: 23, SEQ ID NO: 24, SEQ ID NO: 25, SEQ ID NO: 26, SEQ ID NO: 27, of SEQ ID NO: 29, SEQ ID NO: 30, SEQ ID NO: 31, SEQ ID NO: 33, SEQ ID NO: 34 SEQ ID NO: 35, SEQ ID NO: 36 and SEQ ID NO: 37.

In one embodiment the composition comprises a plurality of immunogenic HSV glycoprotein D peptides. In one embodiment two or more of the plurality of immunogenic HSV glycoprotein D peptides are component parts of one or more fusion proteins or polypeptides.

In one embodiment the composition is a vaccine.

In one embodiment the composition comprises an adjuvant.

In one embodiment, the HSV glycoprotein peptide is an HSV glycoprotein D2 peptide.

In a fourth aspect of the invention there is provided a method for inducing an immune response to HSV in a subject, the method comprising administering to said subject an effective amount of at least one immunogenic HSV glycoprotein D peptide comprising an amino acid sequence selected from the group consisting of SEQ ID Nos 1 to 39 or an immunogenic fragment or variant thereof.

In one embodiment the method comprises administering a pharmaceutical composition comprising at least one immunogenic HSV glycoprotein D peptide, said peptide comprising an amino acid sequence selected from the group consisting of SEQ ID Nos 1 to 39 or an immunogenic fragment or variant thereof, together with a pharmaceutically acceptable carrier, adjuvant or excipient.

In a fifth aspect of the invention there is provided a method for treatment of an HSV infection in a subject, the method comprising administering to said subject a therapeutically effective amount of a composition comprising at least one immunogenic HSV glycoprotein D peptide comprising an amino acid sequence selected from the group consisting of SEQ ID Nos 1 to 39 or an immunogenic fragment or variant thereof.

In a sixth aspect of the invention there is provided a method for prevention of an HSV infection in a subject, the method comprising administering to said subject an effective amount of a composition comprising at least one immunogenic HSV glycoprotein D peptide comprising an amino acid sequence selected from the group consisting of SEQ ID Nos 1 to 39 or an immunogenic fragment or variant thereof.

In one embodiment of the method of the fourth, fifth or sixth aspects the HSV to which an immune response is induced or the infection of which is treated or prevented is HSV2. In one embodiment of the method of the fourth, fifth or sixth aspects the HSV to which an immune response is induced or the infection of which is treated or prevented is HSV1. In one embodiment of the method of the fourth, fifth or sixth aspects the HSV to which an immune response is induced or the infection of which is treated or prevented is HSV1 and HSV2.

In a seventh aspect of the invention there is provided a method for determining the level of T-lymphocyte immunity to HSV1 or 2 in a patient, comprising:

(a) obtaining a biological sample from the patient;

(b) contacting the sample with a peptide comprising an amino acid sequence selected from the group consisting of SEQ ID Nos 1 to 39 or an immunogenic fragment or variant thereof; and

(c) quantifying T-lymphocytes that respond to the peptide.

In an eighth aspect of the invention there is provided a method for detecting HSV infection in a biological sample, comprising:

(a) contacting the biological sample with a binding agent which is capable of binding to an amino acid sequence selected from the group consisting of SEQ ID Nos 1 to 39 or an immunogenic fragment or variant thereof; and

(b) detecting in the sample a peptide that binds to the binding agent.

In a ninth aspect of the invention there is provided a method for determining the level of T-lymphocyte immunity to HSV in a biological sample, comprising:

(a) contacting the biological sample with a binding agent which is capable of binding to an amino acid sequence selected from the group consisting of SEQ ID Nos 1 to 39 or an immunogenic fragment or variant thereof; and

(b) detecting in the sample a peptide that binds to the binding agent; and

(c) quantifying T-lymphocytes that respond to the peptide.

In one embodiment of the eighth and ninth aspects the binding agent is capable of specifically binding to the peptide.

In one embodiment of the eighth and ninth aspects the binding agent is a monoclonal antibody or a polyclonal antibody.

In one embodiment of the seventh aspect the sample is contacted with a plurality of peptides.

In one embodiment of the seventh, eighth and ninth aspects the biological sample is selected from the group consisting of whole blood, serum, plasma, saliva, cerebrospinal fluid and urine.

In one embodiment of the seventh, eighth and ninth aspects the peptide comprises an amino acid sequence selected from the group consisting of SEQ ID NO: 2,

SEQ ID NO: 4, SEQ ID NO: 10, SEQ ID NO: 12, SEQ ID NO: 24, SEQ ID NO: 26, SEQ ID NO: 30, SEQ ID NO: 34 and SEQ ID NO: 36.

In one embodiment of the seventh, eighth and ninth aspects the peptide comprises an amino acid selected from the group consisting of SEQ ID NO:1, SEQ ID NO: 3, SEQ ID NO: 5, SEQ ID NO: 9, SEQ ID NO: 11, SEQ ID NO: 13, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one embodiment of the seventh, eighth and ninth aspects the peptide consists of an amino acid sequence selected from the group consisting of SEQ ID NO: 2, SEQ ID NO: 4, SEQ ID NO: 10, SEQ ID NO: 12, SEQ ID NO: 24, SEQ ID NO: 26, SEQ ID NO: 30, SEQ ID NO: 34 and SEQ ID NO: 36.

In one embodiment of the seventh, eighth and ninth aspects the peptide consists of an amino acid selected from the group consisting of SEQ ID NO:1, SEQ ID NO: 3, SEQ ID NO: 5, SEQ ID NO: 9, SEQ ID NO: 11, SEQ ID NO: 13, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one embodiment of the seventh, eighth and ninth aspects the peptide has binding specificity for at least one class II major histocompatability complex protein.

In one embodiment of the seventh, eighth and ninth aspects the class II major histocompatability complex protein is either or both of:

(a) an HLA-DR protein

(b) an HLA-DQ protein.

In one embodiment of the seventh, eighth and ninth aspects the HLA-DR protein is selected from the group consisting of HLA DRM*0101, HLA DRB1*0301, HLA DRB1*0401, HLA DRB1*0404, HLA DRB1*0405, HLA DRB1*0701, HLA DRB1*1101, HLA DRB1*1302, HLA DRB1*1501 and HLA DRB3*0101.

In one embodiment of the seventh, eighth and ninth aspects the HSV glycoprotein D is HSV glycoprotein D2.

In a tenth aspect the invention provides a diagnostic or prognostic kit comprising at least one component selected from the group consisting of:

(a) an isolated immunogenic Herpes simplex virus (HSV) glycoprotein D peptide comprising an amino acid sequence selected from the group consisting of SEQ ID Nos 1 to 39 or an immunogenic fragment or variant thereof;

(b) a polypeptide comprising at least one peptide according to part (a);

(c) a plurality of peptides according to part (a); and

(d) a tetramer reagent comprising a fragment of an HLA-DR molecule bound to a peptide according to part (a).

In one embodiment, the diagnostic or prognostic kit comprises a peptide comprising an amino acid sequence selected from the group consisting of SEQ ID NO:1, SEQ ID NO: 2, SEQ ID NO: 3, SEQ ID NO: 4, SEQ ID NO: 5, SEQ ID NO: 9, SEQ ID NO: 10, SEQ ID NO: 11, SEQ ID NO: 12, SEQ ID NO: 13, SEQ ID NO: 23, SEQ ID NO: 24, SEQ ID NO: 25, SEQ ID NO: 26, SEQ ID NO: 27, of SEQ ID NO: 29, SEQ ID NO: 30, SEQ ID NO: 31, SEQ ID NO: 33, SEQ ID NO: 34 SEQ ID NO: 35, SEQ ID NO: 36 and SEQ ID NO: 37.

In one embodiment, the diagnostic or prognostic kit comprises a peptide consisting of an amino acid sequence selected from the group consisting of SEQ ID NO:1, SEQ ID NO: 2, SEQ ID NO: 3, SEQ ID NO: 4, SEQ ID NO: 5, SEQ ID NO: 9, SEQ ID NO: 10, SEQ ID NO: 11, SEQ ID NO: 12, SEQ ID NO: 13, SEQ ID NO: 23, SEQ ID NO: 24, SEQ ID NO: 25, SEQ ID NO: 26, SEQ ID NO: 27, of SEQ ID NO: 29, SEQ ID NO: 30, SEQ ID NO: 31, SEQ ID NO: 33, SEQ ID NO: 34 SEQ ID NO: 35, SEQ ID NO: 36 and SEQ ID NO: 37.

In one embodiment, the diagnostic or prognostic kit comprises an HSV glycoprotein D2 peptide.

In an eleventh aspect the invention provides use of at least one immunogenic HSV glycoprotein D peptide comprising an amino acid sequence selected from the group consisting of SEQ ID Nos 1 to 39 or an immunogenic fragment or variant thereof for the manufacture of a medicament for the treatment or prevention of an HSV infection.

In a twelfth aspect the invention provides at least one immunogenic HSV glycoprotein D peptide comprising an amino acid sequence selected from the group consisting of SEQ ID Nos 1 to 39 or an immunogenic fragment or variant thereof for use in the treatment or prevention of an HSV infection.

In one embodiment of the eleventh and twelfth aspects the peptide comprises an amino acid sequence selected from the group consisting of SEQ ID NO:1, SEQ ID NO: 2, SEQ ID NO: 3, SEQ ID NO: 4, SEQ ID NO: 5, SEQ ID NO: 9, SEQ ID NO: 10, SEQ ID NO: 11, SEQ ID NO: 12, SEQ ID NO: 13, SEQ ID NO: 23, SEQ ID NO: 24, SEQ ID NO: 25, SEQ ID NO: 26, SEQ ID NO: 27, of SEQ ID NO: 29, SEQ ID NO: 30, SEQ ID NO: 31, SEQ ID NO: 33, SEQ ID NO: 34 SEQ ID NO: 35, SEQ ID NO: 36 and SEQ ID NO: 37.

In one embodiment of the eleventh and twelfth aspects the peptide consists of an amino acid sequence selected from the group consisting of SEQ ID NO:1, SEQ ID NO: 2, SEQ ID NO: 3, SEQ ID NO: 4, SEQ ID NO: 5, SEQ ID NO: 9, SEQ ID NO: 10, SEQ ID NO: 11, SEQ ID NO: 12, SEQ ID NO: 13, SEQ ID NO: 23, SEQ ID NO: 24, SEQ ID NO: 25, SEQ ID NO: 26, SEQ ID NO: 27, of SEQ ID NO: 29, SEQ ID NO: 30, SEQ ID NO: 31, SEQ ID NO: 33, SEQ ID NO: 34 SEQ ID NO: 35, SEQ ID NO: 36 and SEQ ID NO: 37.

In one embodiment of the eleventh and twelfth aspects the HSV glycoprotein D peptide is an HSV glycoprotein D2 peptide.

In a thirteenth aspect the invention provides a method of producing an immunogenic Herpes simplex virus (HSV) glycoprotein D peptide, the method comprising culturing a host cell comprising an amino acid sequence selected from the group consisting of SEQ ID Nos 1 to 39 or an immunogenic fragment or variant thereof under conditions conducive to the expression of the peptide and optionally isolating the expressed peptide.

In a fourteenth aspect the invention provides an isolated antibody capable of binding specifically to an immunogenic Herpes simplex virus (HSV) glycoprotein D peptide comprising an amino acid sequence selected from the group consisting of SEQ ID Nos 1 to 39 or an immunogenic fragment or variant thereof.

In one embodiment of the thirteenth and fourteenth aspects, the peptide comprises an amino acid sequence selected from the group consisting of SEQ ID NO: 2, SEQ ID NO: 4, SEQ ID NO: 10, SEQ ID NO: 12, SEQ ID NO: 24, SEQ ID NO: 26, SEQ ID NO: 30, SEQ ID NO: 34 and SEQ ID NO: 36.

In one embodiment of the thirteenth and fourteenth aspects, the peptide comprises an amino acid sequence selected from the group consisting of SEQ ID NO:1, SEQ ID NO: 3, SEQ ID NO: 5, SEQ ID NO: 9, SEQ ID NO: 11, SEQ ID NO: 13, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one embodiment of the thirteenth and fourteenth aspects, the peptide consists of an amino acid sequence selected from the group consisting of SEQ ID NO: 2, SEQ ID NO: 4, SEQ ID NO: 10, SEQ ID NO: 12, SEQ ID NO: 24, SEQ ID NO: 26, SEQ ID NO: 30, SEQ ID NO: 34 and SEQ ID NO: 36.

In one embodiment of the thirteenth and fourteenth aspects, the peptide consists of an amino acid sequence selected from the group consisting of SEQ ID NO:1, SEQ ID NO: 3, SEQ ID NO: 5, SEQ ID NO: 9, SEQ ID NO: H, SEQ ID NO: 13, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

In one embodiment of the thirteenth and fourteenth aspects, the HSV glycoprotein D peptide is an HSV glycoprotein D2 peptide.

### ABBREVIATIONS

E:T, effector to target ratio.

HSV, herpes simplex virus.

SBT, Sequencing based typing.

PCR-SSO, polymerase chain reaction-sequence specific oligonucleotide.

gD2, HSV2 glycoprotein D.

SKB, SmithKlineBeecham.

AA, amino acids.

GH, genital herpes.

OH, oral herpes.

### DEFINITIONS

Various published documents, such as patents, patent applications and scientific articles are referred to herein. Where permitted and appropriate the contents of such documents are incorporated in their entirety by cross-reference for the purposes of description.

As used herein and unless otherwise clearly indicated, the term “HSV” includes HSV1 and HSV2.

As used herein the term “immunogenic” when used in the context of a peptide or a composition comprising a peptide will be understood to mean that the peptide is capable of inducing a specific immune response when administered to an organism capable of raising an immune response. It will of course be understood that the degree of immune response to any given peptide or composition comprising a peptide may vary between individuals, such that the immune response raised by one individual to a given peptide may be more or less than the response raised by a second individual administered the same peptide. For the sake of clarity, it is noted that if a peptide is capable in any individual of inducing a specific immune response, then such a peptide will be understood as “immunogenic” as used herein.

As used herein the term “HSV infection” will be understood to encompass any stage of HSV infection, including but not limited to primary HSV infection, recurrent HSV infection and latent HSV infection.

As used herein the term “plurality” means more than one. In certain specific aspects or embodiments, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any integer derivable therein, and any range derivable therein.

As used herein, the terms “antibody” and “antibodies” include IgG (including IgG1, IgG2, IgG3, and IgG4), IgA (including IgA1 and IgA2), IgD, IgE, or IgM, and IgY, whole antibodies, including single-chain whole antibodies, and antigen-binding fragments thereof. Antigen-binding antibody fragments include, but are not limited to, Fab, Fab′ and is F(ab′)2, Fd, single-chain Fvs (scFv), single-chain antibodies, disulfide-5 linked Fvs (sdFv) and fragments comprising either a VL or VH domain. The antibodies may be from any animal origin. Antigen-binding antibody fragments, including single-chain antibodies, may comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies may be monoclonal, polyclonal, chimeric, multispecific, humanized, and human monoclonal and polyclonal antibodies which specifically bind the biological molecule.

In the context of this specification, the term “comprising” means “including principally, but not necessarily solely”. Furthermore, variations of the word “comprising”, such as “comprise” and “comprises”, have correspondingly varied meanings.

Any description of prior art documents herein, or statements herein derived from or based on those documents, is not an admission that the documents or derived statements are part of the common general knowledge of the relevant art in Australia or elsewhere.

### BRIEF DESCRIPTION OF FIGURES

FIG. 1: The amino acid sequences of HSV2 gD and key HSV1 and 2 peptide epitopes for induction of CD4 lymphocyte responses. Each 20 mer peptide analogue had a 10 amino acid overlap with adjacent peptides. Nine 12 mers were synthesized from each 20 mer of peptide 2, 24, 30 and 34 for fine mapping. Each 12 mer overlapped by 11 amino acids with adjacent 12 mer peptides (A). The differences in amino acid sequences between HSV1 (strain 17) and HSV2 (clinical isolate #356.2038) for key peptides tested are shown in (B).

FIG. 2: Peptides of glycoprotein D recognised by CD4 lymphocytes from four HSV2 seropositive patients with recurrent genital herpes. CD4 lymphocytes were enriched by negative selection as outlined in the Methods section herein and stimulated with a UV inactivated HSV2 antigen. Target cells were LCLs incubated with each of the individual peptides or gD2 and with ⁵¹Cr. Exogenous recombinant gD2 was used as the positive control. Effectors and targets were mixed in a ratio of 5:1. Each peptide was tested in triplicate and histograms represent means. Dashed line represents mean of no peptide controls and 3 standard deviations. Key peptide recognition was later checked by IFN-γ Elispot (Methods). Four experiments (i.e. patient 7, 8, 1 and 4) is representative of all donors shown in Table 1A are shown in FIG. 2A, 2B, 2C and 2D respectively.

FIG. 3: Verification of specificity of peptide specific T-cell lines. A CD4 (cytotoxic) lymphocyte cell line from patient 3 was restimulated with peptide 12 through two cycles and then specificity of the cell lines was tested against autologous target PHA blasts with a range of peptides including the peptide stimulator (Kimura and Sasazuki., 1992, Eleventh International Histocompatability Workshop Reference Protocol for the HLA-DNA typing technique, in HLA 1991, Tsuji, Aizawa, and Sasazuki, eds. Oxford University Press, Oxford, p. 397), the overlapping flanking peptides and, as positive controls, target cells incubated with gD2 or infected with HSV2. The effector to target ratio (E:T) was 20:1. Experiments were carried out in triplicate. Representative of 3 experiments with different peptides (and donors) is shown.

FIG. 4: Recognition of gD2 12 mer peptides by CD4 lymphocytes of HSV1+ and HSV2+ patients

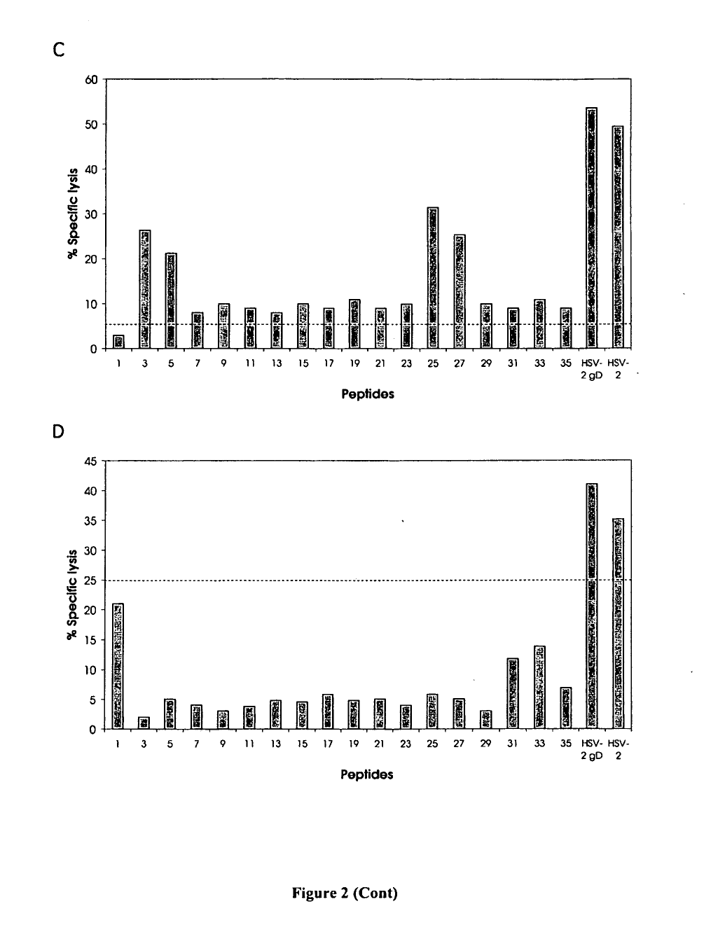

A: IFN-γ production by CD4 lymphocytes of two HSV1−/2+ patients (panels (i) patient 13 and (ii) patient 14) after stimulation with gD2 peptides. The immune response was measured by ELISpot. The dashed line indicates the threshold for recognition which was three standard deviation above mean value of non-stimulated CD4 T cell response (as in FIG. 2).

B: IFN-γ production by CD4 lymphocytes of two HSV1+/2− patients (panels (i) patient 19 and (ii) patient 20) after stimulation with gD2 peptides. The immune response was measured by ELISpot. The dashed line indicates the threshold for recognition which was three standard deviation above mean value of non-stimulated CD4 T cell response (as in FIG. 2).

FIG. 5: Comparison of empirically determined 20 mer and 12 mer peptide epitopes (A) with those predicted from TEPITOPE (5% threshold) (B).

FIG. 6: Immune response profiles of HSV1+ or HSV2+ subject to nine serial gD2 12 mer peptides within immunodominant 20 mers (peptides 2, 24, 30, 34). Mann-Whitney test was performed to obtain y values which are represented as estimated marginal means (see Materials and Methods section herein). Triangle symbols represent HSV 1−/2+ subjects and square symbols HSV1+/2− subjects.

FIG. 7: HLA DR peptide binding assay on HSV glycoprotein D2 peptides. Peptide 2 (SEQ ID NO: 2), peptide 24, (SEQ ID NO: 24), peptide 30 (SEQ ID NO: 30) and peptide 34 (SEQ ID NO: 34) and 12mer peptides derived from each (2.1-2.9—SEQ ID Nos 41-49; 24.1-24.9—SEQ ID Nos 50-58; 30.1-30.9—SEQ ID Nos 59-67; 34.1-34.9 SEQ ID Nos 68-76) were tested for in vitro binding to 10 common HLA DR molecules. A dash indicates 50% inhibitory concentration (IC50)>5000 nM. Significant affinity threshold<1000 are shown in bold type.

### DETAILED DESCRIPTION AND PREFERRED EMBODIMENTS

As described herein, the present inventors have defined immunodominant peptide epitopes recognised by 8 HSV1+ and/or 16 HSV2+ patients, using ⁵¹Cr release cytotoxicity and interferon gamma (IFN-γ) ELISpot assays. Using a set of 39 overlapping 20 mer peptides (Table 1) more than 6 immunodominant epitopes recognised by 12 HSV2+ subjects were defined in glycoprotein gD2, including one each in the leader and transmembrane regions (2-6 peptide epitopes were recognised for each subject). MHC II typing of all 12 subjects showed all were restricted by multiple HLA-DR alleles. One 20 mer (peptide 30) was restricted by multiple HLA-DR and HLA-DQ alleles. Further fine mapping of four of the 20 mers, using a panel of nine internal 12 mers for each 20 mer (Table 2), also demonstrated this promiscuity for 12 mers across multiple HLA-DR alleles. All four 20 mer peptides were cross-recognised by both HSV1+/2- and HSV1−/2+ subjects. In the two 20 mers with the most divergent sequences between HSV1 and 2 the sites of recognition differed within the 20 mers. This work provides a basis for CD4 lymphocyte cross-recognition of gD2 and cross-protection observed in vaccine trials and also provides a reagent or reagents for detecting both HSV1 and 2 specific CD4 lymphocytes simultaneously.

Arising from this work the invention provides, in one aspect, an isolated HSV glycoprotein D peptide. The peptide comprises a fragment of HSV glycoprotein D. Typically, the peptide is an immunogenic peptide.

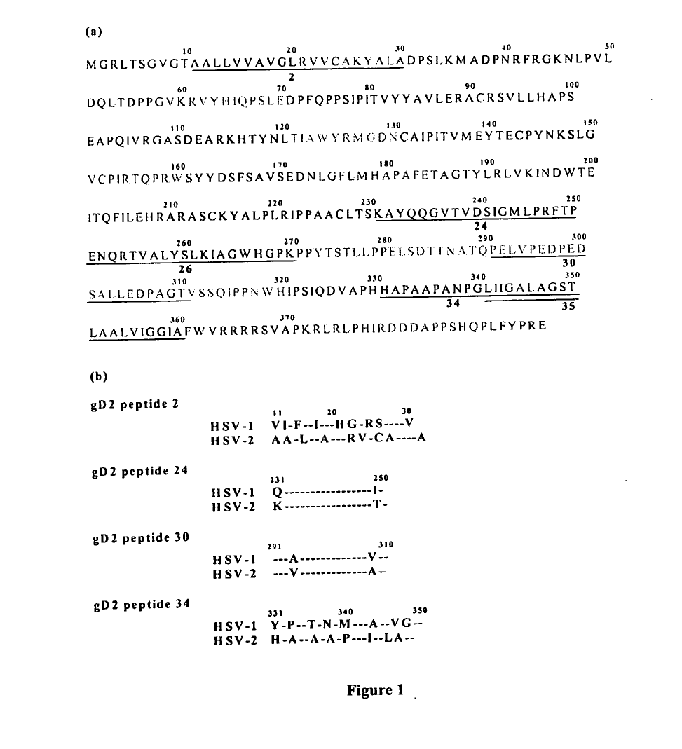

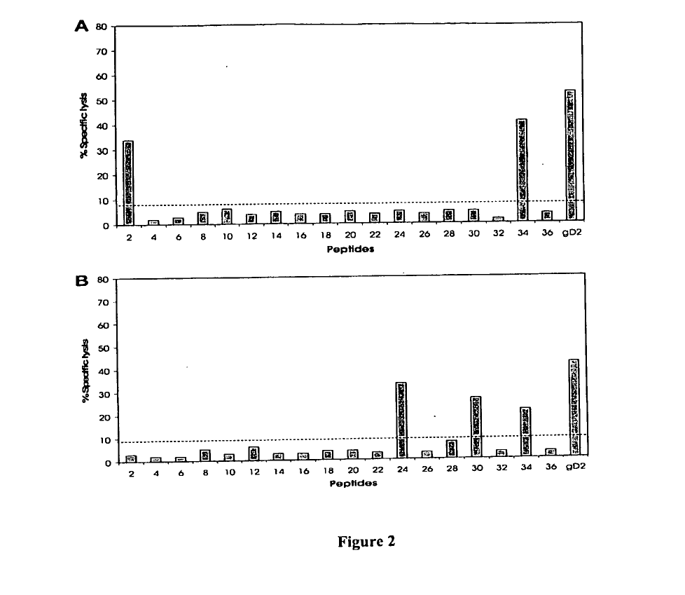

The glycoprotein D peptide is preferably selected from HSV2 glycoprotein D. Alternatively, the glycoprotein D peptide may be selected from homologous regions of HSV1 gD which show high sequence similarity. The full amino acid sequence of HSV2 glycoprotein D from clinical isolate #356.2038 is provided herein in FIG. 1 and Table 1 (including the leader sequence) and is used as the reference sequence for amino acid positions. The skilled addressee will be aware that sequence variations have been described for alternative isolates of HSV2 glycoprotein D and that there exist additional (minor) amino acid variations in the sequence between HSV1 and HSV2 glycoprotein 2. For example, alternative HSV gD2 sequences (e.g. strain HG52) have been described by [Dolan et al., J. Virol., 72:2010, 1998]. Peptides of the invention include corresponding peptides from alternative gD2 sequences. For the sake of clarity, as an example, Peptide 2 (SEQ ID NO: 2) described herein is a 20 mer consisting of the amino acid sequence AALLVVAVGL RVVCAKYALA which corresponds to amino acid positions 11-30 of the gD2 amino acid sequence described herein. It will be understood that peptides of the invention include peptides comprising the corresponding regions and or sequences of other gD sequences, such as other gD2 sequences, such as those referred to above.

It will be understood that the invention encompasses variants of the glycoprotein D peptides described herein. Typically, a variant is a sequence variant. Typically, the variant is an immunogenic peptide such that it retains the ability to elicit an immune response to HSV or HSV-infected cells. Immunogenic variants may be identified by evaluating the reactivity of the peptide using a known assay such as a T cell assay described herein. Variants include those referred to above, in which natural sequence variations occur. Additionally, a variant may be prepared by recombinant or synthetic methods known in the art [Caruthers, M. H. et al. (1980) Nucl. Acids Res. Symp. Ser. 215-223, Horn, T. et al. (1980) Nucl. Acids Res. Symp. Ser. 225-232, Sambrook, J. et al. (1989) Molecular Cloning, A Laboratory Manual, Cold Spring Harbor Press, Plainview, N.Y.].

Variants may have one or more amino acid substitutions, deletions, additions and/or insertions in the amino acid sequence. Peptide variants preferably exhibit at least about 70%, at least about 80%, at least about 85%, or at least about 90% identity to the identified peptide, more preferably at least about 92%, at least about 95% or at least about 97% identity to the identified peptide, and most preferably at least about 97% identity to the identified peptides.

Variants may be modified by, for example, the deletion or addition of amino acids that have minimal influence on the immunogenicity, secondary structure and hydropathic nature of the polypeptide.

Amino acid substitutions include, but are not necessarily limited to, amino acid substitutions known in the art as “conservative”. A “conservative” substitution is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

Typically, a variant peptide differs from a sequence identified herein by substitution, deletion or addition of five amino acids or fewer, such as by four, or three, or two, or one amino acids.

Typically, a peptide of the invention is an isolated peptide. It will be understood that the term “isolated” in this context means that the peptide has been removed from or is not associated with some or all other components with which it would be found in the natural system. For example, an “isolated” peptide may be removed from other amino acid sequences within a gD2 polypeptide sequence, or may be removed from natural components such as unrelated proteins. For the sake of clarity, an “isolated” peptide also includes a peptide which has not been taken from nature but rather has been prepared de novo, such as chemically synthesised and or prepared by recombinant methods. As described herein the isolated peptide of the invention may be included as a component part of a longer polypeptide or fusion protein.

The peptide sequences exemplified herein, such as those in Tables 1 and 2 (SEQ ID Nos: 1-76), consist of 12 amino acids or 20 amino acids. As will be apparent from the Examples and description herein, the peptides listed in the Tables herein and defined by SEQ ID Nos: 1-76) include overlapping regions. It will also be apparent from the results presented herein that further fragments of immunogenic peptides can be identified. For example, it will be recognised that the peptides listed in Table 2 may be described as fragments of the peptides listed in Table 1. As will be recognised by the skilled addressee on the basis of the description herein, an exemplified peptide may further include one or more additional amino acids corresponding to amino acids immediately upstream and/or downstream of the exemplified peptide. As noted above, the skilled addressee will recognise on the basis of the description herein that one or more amino acids of an exemplified peptide herein may be deleted without loss of immunogenic activity.

Accordingly, in preferred embodiments a peptide of the invention may comprise at least about 6 amino acids to at least about 30 amino acids. Preferably, a peptide of the invention may comprise about 7, 8, 9, 10, 11, 12, 13, 14, 15, 16, 17, 18, 19, 20, 21, 22, 23, 24, 25, 26, 27, 28, or 29 amino acids. More preferably, a peptide of the invention may comprise about 12 amino acids or about 20 amino acids. More preferably still, a peptide of the invention may comprise or consist of an amino acid sequence as set out in any of SEQ ID Numbers SEQ ID Nos 1 to 39 or SEQ ID Nos 41-76. Specific peptides of the invention are also listed in Tables 1 and 2 herein SEQ ID Nos 1-76).

Peptides of the invention will, in general, be immunogenic peptides. For example, a peptide of the invention may comprise one or more epitopes capable of being recognized and bound by the immune cells of an organism to which the peptide is administered. Preferably, the immune cells of the host organism capable of recognising and binding the peptide are T lymphocytes.

Preferred, non-limiting examples of 20mer peptides of the invention comprising immunogenic epitopes include those with amino acid sequences defined by SEQ ID Nos: 2, 24, 30 and 34. Other preferred, non-limiting examples of 20mer peptides include those with amino acid sequences defined by SEQ ID Nos: 4, 10, 12, 26, and 36.

It will be recognised that the immunogenic properties of a given HSV glycoprotein D peptides of the invention may extend to overlapping peptide fragments. When a peptide fragment of the invention comprises one or more epitopes capable of being recognised and bound by the immune cells of a given host, some or all of the epitope(s) may be present in an overlapping peptide fragment. Accordingly, the peptide fragment(s) overlapping an immunogenic peptide of the invention may have similar, identical or greater immunogenic properties. For example, the HSV glycoprotein D peptides defined in SEQ ID Nos: 1 and 3 respectively may have immunogenic properties similar to SEQ ID NO; 2. Other non-limiting examples include HSV glycoprotein D peptides which overlap with any of SEQ ID Nos: 4, 10, 12, 24, 26, 30, 34 and 36, being SEQ ID Nos: 5, 9, 11, 13, 23, 25, 27, 29, 31, 33, 35, and 37.

Non-limiting examples of 12mer peptides comprising immunogenic epitopes include those with amino acid sequences defined by SEQ ID Nos: 41-76.

A peptide of the invention may be included as a component part of a longer amino acid sequence. For example, a peptide of the invention may be present within the form of a fusion protein or polypeptide where the peptide is linked with one or more amino acid sequences to which it would not be linked to in nature.

In this context it will be understood that a fusion protein or polypeptide may comprise a plurality of peptides of the invention, such as a polypeptide where two peptides, three peptides, four peptides, five peptides or more of the invention are present on a single polypeptide. In this context, it will be understood that the arrangement of such peptides on the polypeptide or fusion protein will not extend to an arrangement such that the gD2 protein is constituted. Any combination of peptides of the invention may be contemplated. As a non-limiting example, a fusion protein or polypeptide which comprises a plurality of peptides of the invention may include any two, three, four, five or more peptides listed in Table 1 and/or Table 2 herein (SEQ ID Nos 1-76). In preferred embodiments a plurality of peptides may be selected such that the fusion protein comprises peptides identified as comprising advantageous immunogenic responses in a given set of circumstances, as can be determined by the skilled addressee. For example, such a fusion protein or polypeptide may comprise any two or more peptides in any combination selected from the group consisting of SEQ ID Nos 1, 2, 3, 4, 5, 9, 10, 11, 12, 13, 23, 24, 25, 26, 27, 29, 30, 31, 33, 34, 35, 36, and 37.

Peptides of the invention may be expressed as antigens by host immune cells. Typically, peptides of the invention introduced to host immune cells are processed and displayed on the cellular surface bound to Major Histocompatibility Complex (MHC) proteins of the cell. The display of these antigenic determinants in association with the MHC proteins may elicit the proliferation of host immune cells including T-lymphocyte clones specific to the determinants. In humans, MHC proteins are known as Human Leukocyte Antigen (HLA) proteins, and may be class I proteins (HLA A, HLA B or HLA C) or class 11 proteins (HLA DR, HLA DP, or HLA DQ).

Peptides of the invention may be capable of being presented (as an antigen) on and thus specifically binding to any MHC protein. For example, peptides of the invention may be capable of being presented on class II proteins such as HLA-DR, HLA DQ and/or HLA DP. In certain embodiments, the peptides of the invention may be capable of specifically binding multiple different HLA proteins (i.e. multiple different HLA allelic variants). Non-limiting examples of HLA DR allelic variants to which peptides of the invention may be capable of binding include HLA DRB1*0101, HLA DRB1*0301, HLA DRB1*0401, HLA DRB1*0404, HLA DRB1*0405, HLA DRB1*0701, HLA DRB1*1101, HLA DRB1*1302, HLA DRB1*1501 and HLA DRB3*0101.

A fusion protein or polypeptide comprising one or more peptide(s) of the invention may additionally comprise one or more unrelated sequences. In this context it will be understood that an “unrelated sequence” is a sequence which is not present in gD2. Such a sequence will generally be referred to herein, in the context of a fusion protein or polypeptide, as a “fusion partner”. Typically, a fusion partner is an amino acid sequence, and may be a polypeptide. A fusion partner may, for example, be selected to assist with the production of the peptide or peptides. Examples of such fusion partners include those capable of enhancing recombinant expression of the peptide or of a is polypeptide comprising the peptide; those capable of facilitating or assisting purification of the peptide or a polypeptide comprising the peptide such as an affinity tag. Alternatively, or in addition, a fusion partner may be selected to increase solubility of the peptide or of a polypeptide comprising the peptide, to increase the immunogenicity of the peptide, to enable the peptide or polypeptide comprising the peptide to be targeted to a specific or desired intracellular compartment.

Methods for the preparation of fusion proteins are known in the art, for example being described in Chapter 16: Protein Expression, In: Current Protocols in Molecular Biology, Eds Ausubel, F. M. et al, 2007. Typically, a fusion protein may be made by standard techniques such as chemical conjugation, peptide synthesis or recombinant means. A fusion protein may include one or more linker(s), such as peptide linker(s), between component parts of the protein, such as between one or more component peptides, and/or between one or more fusion partners and/or component peptides. Such a peptide linker (s) may be chosen to permit the component parts of the fusion protein to maintain or attain appropriate secondary and tertiary structure.

Peptides of the invention may be prepared by any suitable means, such as by isolation from a naturally occurring form in a gD2 polypeptide or related sequence, by chemical synthesis or by recombinant means. The skilled addressee will be aware of standard methods for such preparation, such as by isolation from a naturally occurring longer amino acid sequence by enzymatic cleavage, such as by chemical synthesis for example as described in Caruthers, M. H. et al. (1980) Nucl. Acids Res. Symp. Ser. 215-223, Horn, T. et al. (1980) Nucl. Acids Res. Symp. Ser. 225-232, such as by recombinant DNA technology for example as described in Sambrook, J. et al. (1989) Molecular Cloning, A Laboratory Manual, Cold Spring Harbor Press, Plainview, N.Y. Chapter 16: Protein Expression, In: Current Protocols in Molecular Biology, Eds Ausubel, F. M. et al, 2007.

A desired peptide, or fusion protein or polypeptide comprising at least one peptide of the invention, may be synthesized, in whole or in part, using chemical methods known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the peptide or protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.).

A synthesized peptide may be purified by preparative high performance liquid chromatography (e.g., Creighton, T. (1983) Proteins, Structures and Molecular Principles, WH Freeman and Co., New York, N.Y.) or other comparable techniques available in the art. If desired, the composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

The peptide of the invention, or a fusion protein or polypeptide comprising a peptide of the invention as a component part thereof may be a soluble peptide, fusion protein or polypeptide.

The invention provides polynucleotides that encode one or more peptide(s) of the invention and polynucleotides that encode one or more fusion protein(s) or polypeptide(s) comprising a peptide(s) of the invention, as described herein. In certain embodiments of the invention, polynucleotide sequences or fragments thereof which encode peptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be recognized by the skilled artisan, polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials. Polynucleotides may comprise a native sequence (i.e., an endogenous sequence that encodes an HSV protein or a portion thereof) or may comprise a variant, or a biological or antigenic functional equivalent of such a sequence. Polynucleotide variants may contain one or more substitutions, additions, deletions and/or insertions, as further described below, preferably such that the immunogenicity of the encoded polypeptide is not diminished, relative to a native HSV protein. The effect on the immunogenicity of the encoded polypeptide may generally be assessed as described herein. The term “variants” also encompasses homologous genes of xenogenic origin.

In order to express a desired polypeptide, the nucleotide sequences encoding the peptide, fusion protein or polypeptide, or functional equivalents, may be inserted into an appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

The invention thus provides vectors comprising a polynucleotide sequence of the invention. In one embodiment the vector may be an expression vector. The invention also provides a host cell comprising a polynucleotide or vector of the invention. The invention also provides methods for the preparation of a peptide of the invention, such a method comprising culturing a host cell comprising a polynucleotide or expression vector of the invention under conditions conducive to expression of the encoded peptide. In one embodiment, the method further comprises purifying the expressed peptide.

Also contemplated by the invention are antibodies which are capable of binding specifically to the polypeptides of the invention. The antibodies may be used to qualitatively or quantitatively detect and analyse one or glycoprotein D polypeptides of the invention or fragments thereof in a given sample. By “binding specifically” it will be understood that the antibody is capable of binding to the target polypeptide or fragment thereof with a higher affinity than it binds to an unrelated protein. For example, the antibody may bind to the polypeptide or fragment thereof with a binding constant in the range of at least 10⁻⁴M to 10⁻¹⁰M. Preferably the binding constant is at least about 10⁻⁵M, or at least about 10⁻⁶M, more preferably the binding constant of the antibody to the glycoprotein D polypeptides of the invention or fragments thereof is at least about 10⁻⁷M, at least about 10⁻⁸M, or at least about 10⁻⁹M or more.

Antibodies of the invention may exist in a variety of forms, including for example as a whole antibody, or as an antibody fragment, or other immunologically active fragment thereof, such as complementarity determining regions. Similarly, the antibody may exist as an antibody fragment having functional antigen-binding domains, that is, heavy and light chain variable domains. Also, the antibody fragment may exist in a form selected from the group consisting of, but not limited to: Fv, Fab, F(ab)2, scFv (single chain Fv), dAb (single domain antibody), chimeric antibodies, bi-specific antibodies, diabodies and triabodies. An antibody ‘fragment’ may be produced by modification of a whole antibody or by synthesis of the desired antibody fragment. Methods of generating antibodies, including, antibody fragments, are known in the art and include, for example, synthesis by recombinant DNA technology. The skilled addressee will be aware of methods of synthesising antibodies, such as those described in, for example, U.S. Pat. No. 5,296,348 and Ausubel F. M. et al. (Eds) Current Protocols in Molecular Biology (2007), John Wiley and Sons, Inc.

Preferably antibodies are prepared from discrete regions or fragments of the glycoprotein D polypeptide of interest. An antigenic portion of a polypeptide of interest may be of any appropriate length, such as from about 5 to about 15 amino acids. Preferably, an antigenic portion contains at least about 5, 6, 7, 8, 9, 10, 11, 12, 13, 14, 15, 16, 17, 18, 19 or 20 amino acid residues.

In the context of this specification reference to an antibody specific to a glycoprotein D polypeptide of the invention includes an antibody that is specific to a fragment of the polypeptide of interest.

Antibodies that specifically bind to a polypeptide of the invention can be prepared, for example, using the purified glycoprotein D polypeptides or their corresponding nucleic acid sequences using any suitable methods known in the art. For example, a monoclonal antibody, typically containing Fab portions, may be prepared using the hybridoma technology described in Harlow and Lane (Eds) Antibodies—A Laboratory Manual, (1988), Cold Spring Harbor Laboratory, N.Y: Coligan, Current Protocols in Immunology (1991); Goding, Monoclonal Antibodies: Principles and Practice (1986) 2nd ed; and Kohler & Milstein, (1975) Nature 256: 495-497. Such techniques include, but are not limited to, antibody preparation by selection of antibodies from libraries of recombinant antibodies in phage or similar vectors, as well as preparation of polyclonal and monoclonal antibodies by immunizing rabbits or mice (see, for example, Huse et al. (1989) Science 246: 1275-1281; Ward et al. (1989) Nature 341: 544-546).

It will also be understood that antibodies of the invention include humanised antibodies, chimeric antibodies and fully human antibodies. An antibody of the invention may be a bi-specific antibody, having binding specificity to more than one antigen or epitope. For example, the antibody may have specificity for one or more glycoprotein D polypeptides or fragments thereof, and additionally have binding specificity for another antigen. Methods for the preparation of humanised antibodies, chimeric antibodies, fully human antibodies, and bispecific antibodies are known in the art and include, for example as described in U.S. Pat. No. 6,995,243 issued Feb. 7, 2006 to Garabedian, et al. and entitled “Antibodies that recognize and bind phosphorylated human glucocorticoid receptor and methods of using same”.

Generally, a sample potentially comprising glycoprotein D polypeptides can be contacted with an antibody that specifically binds the glycoprotein D polypeptide or fragment thereof. Optionally, the antibody can be fixed to a solid support to facilitate washing and subsequent isolation of the complex, prior to contacting the antibody with a sample. Examples of solid supports include, for example, microtitre plates, beads, ticks, or microbeads. Antibodies can also be attached to a ProteinChip array or a probe substrate as described above.

Detectable labels for the identification of antibodies bound to the glycoprotein D polypeptides of the invention include, but are not limited to fluorochromes, fluorescent dyes, radiolabels, enzymes such as horse radish peroxide, alkaline phosphatase and others commonly used in the art, and colorimetric labels including colloidal gold or coloured glass or plastic beads. Alternatively, the marker in the sample can be detected using an indirect assay, wherein, for example, a second, labelled antibody is used to detect bound marker-specific antibody.

Methods for detecting the presence of or measuring the amount of, an antibody-marker complex include, for example, detection of fluorescence, chemiluminescence, luminescence, absorbance, birefringence, transmittance, reflectance, or refractive index such as surface plasmon resonance, ellipsometry, a resonant mirror method, a grating coupler wave guide method or interferometry. Radio frequency methods include multipolar resonance spectroscopy. Electrochemical methods include amperometry and voltametry methods. Optical methods include imaging methods and non-imaging methods and microscopy.

Useful assays for detecting the presence of or measuring the amount of, an antibody-marker complex include, include, for example, enzyme-linked immunosorbent assay (ELISA), a radioimmune assay (RIA), a Western blot assay. These methods are described in, for example, Clinical Immunology (Stites & Terr, eds., 7th ed. 1991) and Methods in Cell Biology: Antibodies in Cell Biology, volume 37 (Alai, ed. 1993); Basic; and Harlow & Lane, supra.

The invention also provides methods for the production of glycoprotein D polypeptides of the invention. Polypeptides of the invention (and fragments and variants thereof) may be produced using techniques generally known in the art. The skilled addressee will appreciate that the invention is not limited by the method of production or purification used and any other method may be used to produce the peptides of the invention.

For example, peptides of the invention may be produced by digestion of a full glycoprotein D polypeptide with one or more proteinases. The digested fragments may be purified by, for example, high performance liquid chromatographic (HPLC) techniques.

Additionally or alternatively, glycoprotein D polypeptides may be synthesized, for example, using conventional liquid or solid phase synthesis techniques.

Glycoprotein D polypeptides of the invention may be produced using standard recombinant protein production techniques. Such techniques may typically involve the cloning of a gene encoding a peptide of the invention or a larger peptide comprising one or more peptides of the invention into an expression vectors or plasmid for subsequent overexpression in a suitable microorganism.

Suitable methods for the construction and use of expression vectors or plasmids for the production of polypeptides of the invention are described, for example, in standard texts such as Sambrook et al., Molecular Cloning: A Laboratory Manual, Cold Spring Harbor, N.Y., 1989, and Ausubel et al., Current Protocols in Molecular Biology, John Wiley and Sons, copyright 2007. Methods for producing recombinant polypeptides are described in detail, for example, in standard texts such as Coligan et al., Current Protocols in Protein Science, (Chapter 5), John Wiley and Sons, Inc., copyright 2007, and Pharmacia Biotech., The Recombinant Protein Handbook 1994, Pharmacia Biotech.

Commonly used expression systems that may be used for the production of recombinant glycoprotein D polypeptides of the invention include, for example, bacterial (e.g. E. coli), yeast (e.g. Saccharomyces cerevisiae Aspergillus, Pichia pastorisis), viral (e.g. baculovirus and vaccinia), cellular (e.g. mammalian and insect) and cell-free systems. Cell-free systems may be also used including eukaryotic rabbit reticuloctye, wheat germ extract systems, and the prokaryotic E. coli cell-free system, using methods described in, for example, Madin et al., 2000. Proc. Natl. Acad. Sci. U.S.A. 97:559-564, Pelham and Jackson, Eur. J. Biochem., 67: 247-256 (1976), Roberts and Paterson, Proc. Natl. Acad. Sci., 70: 2330-2334 (1973), Zubay, Ann. Rev. Genet., 7: 267 (1973), Gold and Schweiger, Meth. Enzymol., 20: 537 (1971), Lesley et al., J. Biol. Chem., 26694): 2632-2638 (1991), Baranov et al., Gene, 84: 463-466 (1989) and Kudlicki et al., Analyt. Biochem., 206: 389-393 (1992).

Purification of glycoprotein D polypeptides of the invention and fragments and variants thereof may be achieved using standard techniques in the art (see Coligan et al., Current Protocols in Protein Science, (Chapter 6), John Wiley and Sons, Inc., copyright 2007). For example, if the recombinant source contains the glycoprotein D polypeptide in is a soluble state, the polypeptide may be isolated using standard methods, often involving column chromatography. Polypeptides of the invention may be genetically engineered to contain various affinity tags or carrier proteins that aid purification. For example, the use of histidine and protein tags engineered into an expression vector containing glycoprotein D polypeptides may facilitate purification by, for example by metal-chelate chromatography (MCAC) under either native or denaturing conditions. Purification of the polypeptides of the invention may also be scaled-up for large-scale production purposes.

The invention also provides compositions comprising one or more peptide(s) of the invention. As described above, in one embodiment the peptide may be in the form of a component part of a fusion protein or polypeptide. The composition may include one peptide of the invention or may include a plurality of peptides of the invention, such as two, or three, or four, or five peptides of the invention. The composition may include a combination of one or more peptide(s) of the invention incorporated as a component part(s) of one or more fusion proteins as described herein and one or more peptide(s) of the invention not so incorporated.

A peptide of the invention may be formulated into a composition, which may be a medicament, for the treatment or prevention of HSV infection.

Typically, the composition is a pharmaceutical composition in which the one or more peptide(s) is formulated with at least one of a pharmaceutically acceptable carrier, adjuvant or excipient.

A composition according to the invention may be suitable for treating or preventing HSV infection. In such circumstances the composition will comprise an immunogenic peptide. In one embodiment, the amino acid sequence of the peptide is selected from the group consisting of SEQ ID Nos: 1, 2, 3, 4, 5, 9, 10, 11, 12, 13, 23, 24, 25, 26, 27, 29, 30, 31, 33, 34 35, 36 and 37. Typically, such a composition will comprise a therapeutically effective amount of the at least one peptide.

A composition according to the invention may comprise any number or combination of immunogenic glycoprotein D peptides. The peptides of the composition may be individual peptides and/or peptides in the form of one or more fusion proteins. The fusion protein or fusion proteins of the composition may comprise any number immunogenic peptides, and may further comprise additional non-related peptides.

The term “therapeutically effective amount” as used herein, includes within its meaning a non-toxic but sufficient amount a compound or composition for use in the invention to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

Generally, the term “therapeutically effective amount” means an amount of said composition or peptide which is capable of inducing an immune response against one or more strains of HSV. Typically, a therapeutically effective amount when administered to a subject will induce an immune response in the subject sufficient to diminish the severity of infection upon subsequent exposure of said subject to a strain or strains of HSV or to diminish one or more symptoms of an HSV infection when administered to an HSV-infected subject. It will be understood that reduction in any one or more symptoms typically seen in HSV infection is encompassed within the meaning, for example a decrease in the duration of infection, a decrease in the duration of one or more symptoms, such as genital or oral lesions, cold sores and infections of mucous membranes, such as gingivostomatitis and keratoconjunctivitis, herpes keratitis, fever blisters, eczema herpeticum, cervical cancer, throat infections, rash, meningitis, and nerve damage, and a decrease in the duration of latency period.

Accordingly, the invention provides methods for the treatment of a condition selected from the group consisting of genital, anal or oral lesions, infections of mucous membranes, such as gingivostomatitis and keratoconjunctivitis, herpes keratitis, fever blisters, eczema herpeticum, cervical cancer, throat infections, rash, meningitis, and nerve damage, where said condition is associated with infection with HSV.

A therapeutically effective amount may be administered to a subject in one dose or may be administered in more than one dose.

Typically, a composition of the invention is a vaccine.

The composition, such as a vaccine, may comprise a polynucleotide encoding one or more peptide(s) of the invention. In this manner administration of the composition to a subject permits the peptide to be generated in situ. The composition may be a DNA vaccine.

In general, suitable compositions may be prepared according to methods which are known to those of ordinary skill in the art and accordingly may include a pharmaceutically acceptable carrier, diluent and/or adjuvant.

The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

These compositions can be administered by standard routes. In general, the compositions may be administered by the parenteral (e.g., intravenous, intraspinal, subcutaneous or intramuscular), oral or topical route. More preferably administration is by the parenteral route.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents. Another type of ‘self adjuvant’ is provided by the conjugation of immunogenic peptides to lipids such as the water soluble lipopeptides Pam3Cys or its dipalmitoyl derivative Pam2Cys. Such adjuvants have the advantage of accompanying the immunogenic peptide into the antigen presenting cell (such as dendritic cells) and thus producing enhanced antigen presentation and activation of the cell at the same time. These agents act at least partly through TOLL-like receptor 2. (Reference Brown L E and Jackson D C, Lipid based self adjuvanting vaccines. Current Drug Delivery, 23:83, 2005).

The composition, such as a vaccine, may include a pharmaceutically acceptable excipient such as a suitable adjuvant. Suitable adjuvants are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

In one embodiment, the adjuvant composition may induce an immune response predominantly of the TH1 type. Suitable adjuvants for use in eliciting a predominantly Th1-type response include, for example, a combination of monophosphoryl lipid A, preferably 3-de-O-acylated monophosphoryl lipid A (3D-MPL) together with an aluminium salt. For example, the composition may be formulated with adjuvant AS04 containing aluminum hydroxide (alum) and 3-O-deacylated monophosphorylated lipid A (MPL) such as described in Thoelen, S., et al., “A prophylactic hepatitis B vaccine with a novel adjuvant system”, Vaccine (2001) 19:2400-2403. Other known adjuvants which preferentially induce a TH1 type immune response include CpG containing oligonucleotides. The oligonucleotides are characterised in that the CpG dinucleotide is unmethylated. Such oligonucleotides are well known and are described in, for example WO 96/02555. Immunostimulatory DNA sequences are also described, for example, by is Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. An adjuvant formulation involving QS21, 3D-MPL and tocopherol in an oil-in-water emulsion is described in WO 95/17210. The adjuvant composition may include a formulation involving QS21, 3D-MPL and tocopherol in an oil in water emulsion such as described in WO 95/17210. In one embodiment the composition comprises the adjuvant Montanide ISA720 (M-ISA-720; Seppic, Fairfield, N.J.), an adjuvant based on a natural metabolizable oil.

Vaccines and compositions of the invention may be prepared according to standard methods, for example as is generally described in Pharmaceutical Biotechnology, Vol. 61 “Vaccine Design—the subunit and adjuvant approach”, edited by Powell and Newman, Plenum Press, 1995; “New Trends and Developments in Vaccines”, edited by Voller et al., University Park Press, Baltimore, Md., U.S.A. 1978.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above is agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The topical formulations of the present invention, comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90° C.-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

The invention provides methods for inducing an immune response to HSV in a subject by administering to said subject an effective amount of at least one peptide of the invention. The peptide of the invention may be administered to the subject in the form of a composition of the invention. In such methods at least one peptide administered will be an immunogenic peptide. Typically, the method comprises a method of immunizing a subject against an HJSV infection.

In one embodiment the invention provides a method of treatment or prevention of an HSV infection in a subject. The method typically comprises administering to the subject a therapeutically effective amount of one or more peptide(s) of the invention. In one embodiment, the amino acid sequence of the peptide is selected from the group consisting of SEQ ID Nos: 1, 2, 3, 4, 5, 9, 10, 11, 12, 13, 23, 24, 25, 26, 27, 29, 30, 31, 33, 34 35, 36 and 37. Administration may be in the form of administering a composition of the invention, a peptide of the invention or a combination of both. The “treatment” includes treatment or prevention of a primary; latent or recurrent HSV infection, including HSV1 and HSV2 infection. In preferred embodiments the treatment is effective against both HSV1 and HSV2.

The “subject” is a mammal, such as any mammal of economic, social or research importance including bovine, equine, ovine, primates, and rodents. Typically the subject is a human. The subject may be infected with HSV, suspected of infection with HSV or at risk of infection with HSV. A subject at risk of infection with HSV may be, for example, a sexual of an individual infected with HSV.

In certain embodiments, methods for vaccination include administering a priming dose of a peptide or composition of the invention. The priming dose may be followed by a boost dose. In various embodiments, the peptide or composition is administered at least once, twice, three times or more.

In certain embodiments the methods of the invention include genetic vaccination, also known as DNA immunization, comprising administering a polynucleotide or an expression vector(s) encoding a peptide of the invention in vivo, in vitro, or ex vivo to induce the production of a correctly folded antigen(s) within an appropriate organism, tissue, cell or a target cell(s). The introduction of the genetic vaccine will cause an antigen to be expressed within those cells, an antigen typically being part or all of one or more protein or proteins of a pathogen. The processed proteins will typically be displayed on the cellular surface of the transfected cells in conjunction with the Major Histocompatibility Complex (MHC) antigens of the normal cell. The display of these antigenic determinants in association with the MHC antigens is intended to elicit the proliferation of cytotoxic T-lymphocyte clones specific to the determinants. Furthermore, the proteins released by the expressing transfected cells can also be picked up, internalized, or expressed by antigen-presenting cells to trigger a systemic humoral antibody responses.

The present invention includes methods of immunizing, treating or vaccinating a subject by contacting the subject with an antigenic composition comprising a peptide of the invention or a polynucleotide(s) encoding a peptide of the invention. In one embodiment, the amino acid sequence of the peptide is selected from the group consisting of SEQ ID Nos: 1, 2, 3, 4, 5, 9, 10, 11, 12, 13, 23, 24, 25, 26, 27, 29, 30, 31, 33, 34 35, 36 and 37. An antigenic composition may comprise a nucleic acid; a polypeptide; an attenuated pathogen, such as a virus, a bacterium, a fungus, or a parasite, which may or may not express a peptide of the invention; a prokaryotic cell expressing a peptide of the invention; a eukaryotic cell expressing a peptide of the invention; a virosome; and the like, or a combination thereof. As used herein, an “antigenic composition” will typically comprise an antigen in a pharmaceutically acceptable formulation.

The peptides of the present invention may be administered as a single agent therapy or in addition to an established therapy, such as inoculation with live, attenuated, or killed virus, or any other therapy known in the art to treat HSV or another epitope-sensitive condition. The peptides of the invention may be used administered in conjunction with one or more antigenic peptides or polypeptides which are not derived from HSV glycoprotein D. Where two or more therapeutic entities are administered to a subject “in conjunction”, they may be administered in a single composition at the same time, or in separate compositions at the same time or in separate compositions separated in time. Where the peptide of the invention is used in addition to an alternative peptide or polypeptide-based therapy, the peptide of the invention and the alternative therapy peptide or polypeptide may form component parts of a single polypeptide or fusion protein.

The appropriate dosage of the peptides of the invention may depend on a variety of factors. Such factors may include, but are in no way limited to, a patient's physical characteristics (e.g., age, weight, sex), whether the compound is being used as single agent or adjuvant therapy, the type of MHC restriction of the patient, the progression (i.e., pathological state) of the HSV infection or other epitope-sensitive condition, and other factors that may be recognized by one skilled in the art. In general, a peptide or to combination of peptides may be administered to a patient in an amount of from about 50 micrograms to about 5 mg; dosage in an amount of from about 50 micrograms to about 500 micrograms is especially preferred.

The invention also provides methods for the diagnosis of or detection of HSV in a sample. In one embodiment the method is for detection or diagnosis of HSV infection or level of (T-lymphocyte) immunity in a sample. The sample may be a biological sample which may or may not be suspected of containing HSV specific CD4 lymphocytes. In one embodiment the biological sample is selected from the group consisting of whole blood, cerebrospinal or genital fluids. The sample may be an in vitro sample such as a laboratory experimental sample, such as may be utilised in research applications.

In one embodiment the method is for detecting HSV infection in a patient, comprising: (a) obtaining a biological sample from the patient; (b) contacting the sample with a peptide of the invention; and (c) combining this with fluorescent reagents which detect and quantify CD4 (T) lymphocytes which specifically recognise the peptide. In one embodiment the sample is contacted with a plurality of peptides of the invention.

In one embodiment, the amino acid sequence of the peptide is selected from the group consisting of SEQ ID Nos: 1, 2, 3, 4, 5, 9, 10, 11, 12, 13, 23, 24, 25, 26, 27, 29, 30, 31, 33, 34 35, 36 and 37.

In one embodiment the method is for detecting HSV infection or the level of immunity to HSV in a biological sample, comprising: (a) contacting the biological sample with a binding agent which is capable of binding to a peptide of the invention; and (b) thereby detecting CD4 lymphocytes in the sample which recognise HSV infection.

In one embodiment, the amino acid sequence of the peptide is selected from the group consisting of SEQ ID Nos: 1, 2, 3, 4, 5, 9, 10, 11, 12, 13, 23, 24, 25, 26, 27, 29, 30, 31, 33, 34 35, 36 and 37.

The methods described herein, for example the methods for detecting HSV-specific CD4 T lymphocytes may be used for predicting the likelihood or frequency of a recurrence of herpes disease in an infected subject. Furthermore, the methods may be of use in predicting the likelihood of co-infection or super-infection with additional viruses, including for example the human immunodeficiency virus (HIV).

The invention also provides a diagnostic or prognostic kit comprising at least one component selected from the group consisting of: (a) a peptide of the invention; (b) a polypeptide comprising at least one peptide of the invention; (c) a plurality of peptides of the invention; and (d) a “tetramer” reagent comprising a fragment of an HLA-DR molecule bound to a peptide of the invention.

In one embodiment, the amino acid sequence of the peptide is selected from the group consisting of SEQ ID Nos: 1, 2, 3, 4, 5, 9, 10, 11, 12, 13, 23, 24, 25, 26, 27, 29, 30, 31, 33, 34 35, 36 and 37.

The diagnostic or prognostic kit may further comprise instructions for use of the kit and or reagents in a diagnostic test. Optionally, the kit further comprises one or more detection reagent(s). For example, the kit may comprise streptavidin and or biotin. Optionally, the biotin may be conjugated to a fluorophore, for example phycoerythrin. In one embodiment the kit comprises a fluorescent detection reagent for recognising CD4 lymphocytes bound to the peptide consisting of biotin and a fluorophore such as phycoerythrin. Optionally, one or more of (a), (b), (c), or (d) may comprise or be bound to a detection agent, such as streptavidin.

In one embodiment the tetramer may be a MHCII tetramer.

The kit may further comprise additional reagents, such as buffers.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

In order that the present invention may be more clearly understood preferred forms will be described with reference to the following examples and drawings.

### Materials and Methods

### Patients and HSV Type Specific Serotyping

Blood was obtained from 16 HSV2 seropositive (HSV1−/2+ or HSV1+/2+) patients usually 1-6 months after recurrences of genital herpes and 8 patients who were only HSV1 seropositive (HSV1+/HSV2−) usually 1-12 months after recurrence of oral herpes. Informed consent was obtained from all the blood donors and the study was approved by the Western Sydney Area Health Service Research and Ethics Committee. HSV2 type specific serology was performed by ELISA (Focus Technologies, Cypress, Calif.) and confirmed by Western Blot. HSV1 specific serology was determined by is Western Blot (Ho et al., 1993, Detection of immunoglobulin M antibodies to glycoprotein G-2 by wester blot (immunoblot) for diagnosis of initial herpes simplex virus 2 genital infections, J Clin Microbiol 31: 3157; Cunningham et al., 2006, Prevalence of infection with herpes simplex virus types 1 and 1 in Australia: a nationwide population based survey, Sex Transm Infect 82: 164).

### Molecular MHC II Typing

HLA-DRB1 generic level typing was performed by polymerase chain reaction-sequence specific oligonucleotide (PCR-SSO) typing based on the method described in the 11 th International Histocompatibility Workshop (Kimura and Sasazuki., 1992, Eleventh International Histocompatability Workshop Reference Protocol for the HLA-DNA typing technique, in HLA 1991, Tsuji, Aizawa, and Sasazuki, eds. Oxford University Press, Oxford, p. 397).

Sequencing based typing (SBT) was used for HLA DQB1 and to resolve any ambiguities in HLA types involving HLA DRB1*03, *08, *11, *12, *13 or *14 alleles. PCR product was sequenced in both forward and reverse directions using a BigDye Terminator Kit (Applied Biosystems, Foster City, Calif.) on an ABI3100 sequencer (Applied Biosystems). Sequence data was analyzed using MatchTools sequencing analysis software (Applied Biosystems).

### Herpes Simplex Viruses and Peptides

HSV-2 strain 186 was grown and titred in Vero cells for subsequent use as control.

Initial screening was performed using a library of HSV2 glycoprotein D (gD2) 20 mer peptides. Each of these 39 peptides was 20 amino acids (AA) long and had 10 residue overlaps (Table 1). They were dissolved in DMSO to a final volume of 2 mg/ml and stored at −80° C.

TABLE 1
20 mer peptide sequences of HSV2 glycoprotein D

| Peptide | | | SEQ ID |
|---|---|---|---|
| No. | Sequence | Position | NO. |
| 1 | MGRLTSGVGT AALLVVAVGL | 1-20 | 1 |
| 2 | AALLVVAVGL RVVCAKYALA | 11-30 | 2 |
| 3 | RVVCAKYALA DPSLKMADPN | 21-40 | 3 |
| 4 | DPSLKMADPN RFRGKNLPVL | 31-50 | 4 |
| 5 | RFRGKNLPVL DQLTDPPGVK | 41-60 | 5 |
| 6 | DQLTDPPGVK RVYHIQPSLE | 51-70 | 6 |
| 7 | RVYHIQPSLE DPFQPPSIPI | 61-80 | 7 |
| 8 | DPFQPPSIPI TVYYAVLERA | 71-90 | 8 |
| 9 | TVYYAVLERA CRSVLLHAPS | 81-100 | 9 |
| 10 | CRSVLLHAPS EAPQIVRGAS | 91-110 | 10 |
| 11 | EAPQIVRGAS DEARKHTYNL | 101-120 | 11 |
| 12 | DEARKHTYNL TIAWYRMGDN | 111-130 | 12 |
| 13 | TIAWYRMGDN CAIPITVMEY | 121-140 | 13 |
| 14 | CAIPITVMEY TECPYNKSLG | 131-150 | 14 |
| 15 | TECPYNKSLG VCPIRTQPRW | 141-160 | 15 |
| 16 | VCPIRTQPRW SYYDSFSAVS | 151-170 | 16 |
| 17 | SYYDSFSAVS EDNLGFLMHA | 161-180 | 17 |
| 18 | EDNLGFLMHA PAFETAGTYL | 171-190 | 18 |
| 19 | PAFETAGTYL RLVKINDWTE | 181-200 | 19 |
| 20 | RLVKINDWTE ITQFILEHRA | 191-210 | 20 |
| 21 | ITQFILEHRA RASCKYALPL | 201-220 | 21 |
| 22 | RASCKYALPL RIPPAACLTS | 211-230 | 22 |
| 23 | RIPPAACLTS KAYQQGVTVD | 221-240 | 23 |
| 24 | KAYQQGVTVD SIGMLPRFTP | 231-250 | 24 |
| 25 | SIGMLPRFTP ENQRTVALYS | 241-260 | 25 |
| 26 | ENQRTVALYS LKIAGWHGPK | 251-270 | 26 |
| 27 | LKIAGWHGPK PPYTSTLLPP | 261-280 | 27 |
| 28 | PPYTSTLLPP ELSDTTNATQ | 271-290 | 28 |
| 29 | ELSDTTNATQ PELVPEDPED | 281-300 | 29 |
| 30 | PELVPEDPED SALLEDPAGT | 291-310 | 30 |
| 31 | SALLEDPAGT VSSQIPPNWH | 301-320 | 31 |
| 32 | VSSQIPPNWH IPSIQDVAPH | 311-330 | 32 |
| 33 | IPSIQDVAPH HAPAAPANPG | 321-340 | 33 |
| 34 | HAPAAPANPG LIIGALAGST | 331-350 | 34 |
| 35 | LIIGALAGST LAALVIGGIA | 341-360 | 35 |
| 36 | LAALVIGGIA FWVRRRRSVA | 351-370 | 36 |
| 37 | FWVRRRRSVA PKRLRLPHIR | 361-380 | 37 |
| 38 | PKRLRLPHIR DDDAPPSHQP | 371-390 | 38 |
| 39 | DDDAPPSHQP LFYPRE | 381-396 | 39 |

The complete sequence of gD2 is shown in FIG. 1 (SEQ ID NO:40). For the second screening, the selected peptides based on the result of the first screening were truncated into nine serial 12 mers within the four most frequently recognised 20 mer peptides (peptides 2, 24, 30, 34 shown in Table 1 and FIG. 1). 20 mer peptides (peptides 26 and 35) were tested as well. The selected immunodominant 20 mers and serial 12 mers were produced by Mimotopes (Melbourne, Australia). They had an 11 amino acid sequence overlap with adjacent peptides. The nine serial 12 mers from the four most recognised 20 mers are shown in Table 2. The peptides were dissolved in DMSO at a concentration of 10 mM and stored at −80° C.

TABLE 2
Serial 12 mer peptide sequences.

| Peptide | | | |
|---|---|---|---|
| No. | Sequence | Position | SEQ ID NO. |
| 2 | AALLVVAVGLRVVCAKYALA | 11-30 | 2 |
| 2-1 | AALLVVAVGLRV | | 41 |
| 2-2 | ALLVVAVGLRVV | | 42 |
| 2-3 | LLVVAVGLRVVC | | 43 |
| 2-4 | LVVAVGLRVVCA | | 44 |
| 2-5 | VVAVGLRVVCAK | | 45 |
| 2-6 | VAVGLRVVCAKY | | 46 |
| 2-7 | AVGLRVVCAKYA | | 47 |
| 2-8 | VGLRVVCAKYAL | | 48 |
| 2-9 | GLRVVCAKYALA | | 49 |
| 24 | KAYQQGVTVDSIGMLPRFTP | 231-250 | 24 |
| 24-1 | KAYQQGVTVDSI | | 50 |
| 24-2 | AYQQGVTVDSIG | | 51 |
| 24-3 | YQQGVTVDSIGM | | 52 |
| 24-4 | QQGVTVDSIGML | | 53 |
| 24-5 | QGVTVDSIGMLP | | 54 |
| 24-6 | GVTVDSIGMLPR | | 55 |
| 24-7 | VTVDSIGMLPRF | | 56 |
| 24-8 | TVDSIGMLPRFT | | 57 |
| 24-9 | VDSIGMLPRFTP | | 58 |
| 30 | PELVPEDPEDSALLEDPAGT | 291-310 | 30 |
| 30-1 | PELVPEDPEDSA | | 59 |
| 30-2 | ELVPEDPEDSAL | | 60 |
| 30-3 | LVPEDPEDSALL | | 61 |
| 30-4 | VPEDPEDSALLE | | 62 |
| 30-5 | PEDPEDSALLED | | 63 |
| 30-6 | EDPEDSALLEDP | | 64 |
| 30-7 | DPEDSALLEDPA | | 65 |
| 30-8 | PEDSALLEDPAG | | 66 |
| 30-9 | EDSALLEDPAGT | | 67 |
| 34 | HAPAAPANPGLIIGALAGST | 331-350 | 34 |
| 34-1 | HAPAAPANPGLI | | 68 |
| 34-2 | APAAPANPGLII | | 69 |
| 34-3 | PAAPANPGLIIG | | 70 |
| 34-4 | AAPANPGLIIGA | | 71 |
| 34-5 | APANPGLIIGAL | | 72 |
| 34-6 | PANPGLIIGALA | | 73 |
| 34-7 | ANPGLIIGALAG | | 74 |
| 34-8 | NPGLIIGALAGS | | 75 |
| 34-9 | PGLIIGALAGST | | 76 |

### Preparation of HSV2 Specific CD4 Lymphocyte Effectors

Peripheral blood mononuclear cells (PBMCs) prepared by Ficoll-Hypaque gradient were stimulated with UV-inactivated HSV2 (Kimura and Sasazuki., 1992, Eleventh International Histocompatability Workshop Reference Protocol for the HLA-DNA typing technique, in HLA 1991, Tsuji, Aizawa, and Sasazuki, eds. Oxford University Press, Oxford, p. 397) and then cultured in RPMI 1640 (Invitrogen, Auckland, NZ) supplemented with 10% fetal calf serum (FCS; Invitrogen), 2 mM glutamine (Sigma-Aldrich, St. Louis, Mo.; RF10), and 20 U recombinant IL-2 (Roche, Sydney, Australia). CD4 T cell effectors were enriched by CD8 beads (Miltenyi Biotech, Bergisch Gladbach, Germany) immediately prior to the cytotoxicity experiment. The efficacy of CD8 T-cell depletion was checked routinely by flow cytometry using anti Leu 2a+2b antibody (Becton Dickinson, Sydney, Australia) and showed <1% of CD8 T cell contamination. Peptide-specific CD4 effectors were, if necessary, restimulated with γ-irradiated (5,000 rads) and peptide sensitized (2 μg/ml for 1 hour at 37° C.) autologous PBMC.

### Preparation of Target Cells for Cytotoxicity Assays

B-LCLs were established by EBV transformation of peripheral B cells and used as target cells for ⁵¹Cr release assay, and the HLA DR and DQ blocking assay. PHA blasts prepared as described previously (Mikloska et al., 1996, Herpes simplex virus protein targets with interferon-gamma, J Infect Dis 173: 7) were used to examine the specificity of gD2 peptide-specific CD4 effector T cells. For each of gD2 peptides tested, 10⁴ LCLs were sensitised with 3 μg/ml of peptide and 1 μCi of sterile ⁵¹Cr (Amersham Pharmacia Biotech, UK) added for 90 minutes at 37° C. in 5% CO₂. After sensitisation, the cells were washed three times by centrifugation for 7 min at 270×g with lukewarm RF10 prior to co-culture with effector cells. The two positive control tubes included target cells sensitised with the same concentration of gD2 antigen and target cells infected with 10 PFU/tube of HSV2 instead of peptide. The controls were cell control (non-sensitised targets with effectors), spontaneous release (targets with no effectors), and total release control tubes containing only target cells labelled with ⁵¹Cr sodium solution.

### Chromium Release Cytotoxicity Assays

A total of 10⁴ peptide-sensitised target cells were co-cultured with CD4 T cell effectors in each well of a Lumaplate (PerkinElmer Life & Analytical Sciences, Shelton, Conn.) at E:T ratios of 5:1 and 25:1 for 14 hrs at 37° C. in 5% CO₂ in triplicate. The plates were prepared for analysis on a Packard Topcount™ gamma counter (see DeFreitas et al., 1985, Human T-lymphocyte response in vitro to synthetic peptides of herpes simplex virus glycoprotein D, Proc Natl Acad Sci USA 82: 3425; Brynestad et al., 1990, Influence of peptide acylation, liposome incorporation, and synthetic immunomodulators on the immunogenicity of a 1-23 peptide of glycoprotein D of herpes simplex virus: implications for subunit vaccines, J. Virol 64: 680). The amount of ⁵¹Cr released was quantified as counts per minute (cpm) using a gamma counter and the percentage of specific cytotoxic activity calculated using the following equation:

% specific lysis=[(experimental release−spontaneous release)×100]/(total release−spontaneous release).

Standard errors of experimental cpm (triplicates) were less than 3%. Differences between the percentage of specific ⁵¹Cr release obtained with peptides were assessed for statistical significance by student t-test with P<0.05 indicating recognition of peptide epitopes.

### Interferon Gamma ELISpot Assays

In order to examine the immune response of CD4 T lymphocytes to truncated gD2 peptides, CD8 lymphocytes were depleted from isolated PBMCs using Miltenyi CD8 microbeads (Miltenyi Biotech) according to the manufacturer's instructions. IFN-γ production was measured as the immune response following stimulating CD8 depleted PBMC with 10 μM peptide by ELISpot assay as described below.

A Millipore Plate with immunobilon-P PVDF membrane (Millipore, Bedford, Mass.) was coated with purified IFN-γ capture antibody (1D1K, Mabtech, Mosman, Australia) to a final concentration of 5 μg/ml in sterile PBS. The plate was washed three times with sterile phosphate buffered saline (PBS) and blocked with RF10 for 2 hours. After washing the plate three times with PBS, 5-7×10⁴ CD8 lymphocyte depleted PBMCs were added to each well in 100 μl of RFIO supplemented with 10 ng/ml IL-12. Peptides, UV-inactivated HSV1 and 2, and PHA were added at a final concentration of 10 μM, 0.5 MOI, and 0.5 μg/ml, respectively. After incubating the cells for 40 hours at 37° C., the plate was washed three times with PBS and then three times with PBS containing 0.05% Tween 20 (PBST). Biotinylated IFN-γ detection antibody (Mabtech, 7-B6-1) diluted to 1 μg/ml in PBST containing 1% bovine serum albumin (BSA) was added to each well. The plate was incubated for 2 hours at room temperature and washed six times with PBST. Streptavidin-alkaline phosphatase enzyme conjugate (Bio-Rad, Hercules, Calif.) diluted in 1:1000 was added to each well. After incubating for 45 min at room temperature, the plate was washed three times with PBST, and then four times with PBS. BCIP/NBT (Bio-Rad) was added as substrate according to the manufacturer's instructions followed by incubation for about 5 min at room temperature in the dark. The reaction was stopped by extensive rinsing of the plate under running water with underdrain removed. The plate was dried overnight in the dark. The spots were counted after scanned by KS Elispot System (Zeiss, North Ryde, Australia) and counted manually (see Wilkinson et al., 2002, Identification of Kaposi's sarcoma-associated herpesvirus (KSHV)-specific cytotoxic T-lymphocyte epitopes and evaluation of reconstitution of KSHV-specific responses in human immunodeficiency virus type 1-infected patients receiving highly active antiretroviral therapy, J. Virol 76: 2634).

### In Vitro HLA DR Peptide Binding Assays

Frequently targeted peptides were tested for in vitro binding to 10 common HLA DR molecules. HLA DR molecules were purified, and binding assays were performed essentially as previously described (39). Purified human HLA DR molecules were incubated with unlabeled gD peptides and 0.1-1 nM ¹²⁵I-radiolabeled probe peptides for 48 hours. MHC binding of the radiolabeled peptide was determined by capturing MHC-peptide complexes on LB3.1 (anti-HLA DR) Ab-coated Lumitrac 600 plates (Greiner Bioone) and measuring bound cpm using the TopCount (Packard Instrument) microscintillation counter. The binding data were analyzed and IC₅₀ (nanomolar) determined as previously described (Southwood et al., 1998. Several common HLA-DR types share largely overlapping peptide binding repertoire, J. Immunol. 160: 3363; Wilson et al., 2001, Identification and antinenicity of broadly cross-reactive and conserved human immunodeficiency virus type 1-derived helper T-lymphocyte epitopes, J. Virol 75: 4195).

### T-Cell Epitope Prediction

The whole gD2 sequence was loaded into the peptide prediction software (TEPITOPE) to predict promiscuous epitopes. The prediction threshold was set at 5% and all the available MHC II molecules were selected to match with predicted epitopes (Bian and Hammer, 2003, The use of bioinformatics for identifying class 11-restricted T-cell epitopes, Methods 29:299).

### Statistical Analysis of Type Specific Peptide Epitopes

The statistical software package SPSS for Windows Version 14 was used to analyse the data. For each of the 4 peptides (2, 24, 30 and 34), the CD4 lymphocyte responses by IFN-γ production to 9 internal peptides were available on each subject. The distributions of these results clearly departed from normality for each peptide. Accordingly, rank scores (1 to 9) of the internal peptides were computed for each peptide within each subject. Statistical analyses were performed separately for each peptide using these rank scores.

There were 4 subjects positive only for HSV2 and 8 subjects positive only for HSV1. Repeated measures analysis of variance was used to test whether there was a significant interaction between the effect of internal peptide number (1 to 9) and HSV status (HSV1 only or HSV2 only) on the rank scores for each peptide. Internal peptide number was treated as a within subject factor and HSV status as a between subject factor. Statistical tests of interaction typically require larger sample sizes to achieve adequate power compared to tests of main effects. Since there were only a total of 12 subjects available, a p-value for interaction<0.1 was considered to be statistically significant.

For each peptide, the amino acid sequences of the 9 internal peptides were compared between HSV1 and HSV2. In this way internal peptides with at least two major differences in (unlike) amino acids were identified. If a statistically significant interaction was detected between the effects of internal peptide number (1 to 9) and HSV status, a Mann-Whitney test was then used to compare the rank scores between the HSV groups for those internal peptides identified as having major amino acid discrepancies between HSV1 and HSV2. Two-tailed tests with a 5% significance level were used for these comparisons.

### Results

### Subjects

Overall, 16 patients with a history of genital herpes (GH) and 8 patients with oral herpes (OH) were studied in two stages: general screening for gD2 20 mer peptide recognition in 12 patients with genital herpes and then specific studies in 4 with GH and 8 with OH. A history of recent or current active lesions was noted and then their blood screened by ELISA and/or Western blot for HSV1 and 2 antibodies. In this study all patients with genital herpes were HSV2 seropositive and all patients with oral herpes were HSV1 seropositive. In studies of HSV1/2 cross reactive T-cell epitopes only HSV1+/2− or 1−/2+ patients were used; after screening HSV1+/2+ patients were excluded to avoid ambiguity in interpretation of HSV1/2 cross reactive epitopes. (Table 3A and 3B).

TABLE 3
gD2 peptide recognition by CD4 lymphocytes of HSV1 or
2 seropositive subjects and correlation of MHC II type
A: Recognition of 20 mer peptides by CD4 lymphocytes of
12 HSV2 seropositive patients with recurrent genital herpes

| | | Peptides recognised |
|---|---|---|
| Patient | MHC II alleles | (highest to lowest) |
| 1 | DRB1* 01, 11, DQB1* 05, 07 | 10 = 34 > (3)4(5), 25 = 26 > 27 |
| 2 | DRB1* 01, 04, DQB1* 05, 08 | 34 > 22, 2 |
| 3 | DRB1* 07, 07, DQB1* 02, 02 | 12 > 24 = 30 |
| 4 | DRB1* 04, 11, DQB1* 07, 08 | 2(1), (33)34(35) |
| 5 | DRB1* 01, 15, DQB1* 05, 06 | 35 (34)/36 > 2 |
| 6 | DRB1* 01, 15, DQB1* 06, 10 | 1, 2, 3, 4 |
| 7 | DRB1* 01, 10, DQB1* 02, 05 | 34 > 2 |
| 8 | DRB1* 04, 07, DQB1* 07, 07 | 24 > 30 > 34 |
| 9 | DRB1* 01, 10, DQB1* 01, 07 | 26 > 10 > 4 |
| 10 | DRB1* 04, 04, DQB1* 08, 10 | 24 > 30 > 12 |
| 11 | DRB1* 01, 12, DQB1* 01, 07 | 33, 34 > 24 > 1, 2 |
| 12 | DRB1* 01, 15, DQB1* 03, 07 | 1, 2, 3 > 35, 36 |

B: gD2 12 mer recognition by HSV1 and 2 seropositive subjects

| | | | Recognised gD2 peptides | Parental 20 |
|---|---|---|---|---|
| Patient | HSV serotype | HLA type | (highest to lowest) | mer peptide |
| 13 | HSV1−/2+ | DRB1*04011, 13021, | 2-5, 2-4, 2-3, 2-2, 2-1, 2 | 2 |
| | | DQB1*0302, 0609 | | 24 |
| | | | 30 | 30 |
| | | | 34-1, 34-3, 34-2 | 34 |
| 14 | HSV1−/2+ | DRB1*13011, 1305, | 2-2, 2 | 2 |
| | | DQB1*0301, 0603 | 24-4, 24-5 | 24 |
| | | | 30-6, 30-2, 30-4, 30-5, 30-7 | 30 |
| | | | 34-9 | 34 |
| 15 | HSV1−/2+ | DRB1*0404, 15011, | 2-3, 2-5, 2, 2-9 | 2 |
| | | DQB1*0302, 0602 | 24-4, 24-1, 24, 24-3 | 24 |
| | | | 30-6 | 30 |
| | | | 34-3 | 34 |
| 16 | HSV1−/2+ | DRB1*03011, 04011, | | 2 |
| | | DQB1*0201, 0301 | | 24 |
| | | | | 30 |
| | | | 34-3, 34-2, 34-6 | 34 |
| 17 | HSV1+/2− | DRB1*0404, 13011, | | 2 |
| | | DQB1*0302, 0603 | 24-2, 24-3, 24-4 | 24 |
| | | | 30-4, 30-3 | 30 |
| | | | | 34 |
| 18 | HSV1+/2− | DRB1*0404, 15011, | | 2 |
| | | DQB1*0302, 0602 | | 24 |
| | | | 30-5, 30, 30-6, 30-3 | 30 |
| | | | 34-7 | 34 |
| 19 | HSV1+/2− | DRB1*03011, 0406, | 2-3 | 2 |
| | | DQB1*0201, 0302 | 24-2, 24-4, 24-9 | 24 |
| | | | 30-7, 30-5 | 30 |
| | | | 34-6, 34-5, 34-3, 34-7 | 34 |
| 20 | HSV1+/2− | DRB1*11011, 11041, | 2-3 | 2 |
| | | DQB1*0301, 0301 | 24-4, 24-1, 24-5, 24, 24-3, 24-2 | 24 |
| | | | 30-5 | 30 |
| | | | 34.7, 34.8 | 34 |
| 21 | HSV1+/2− | DRB1*01011, 07011, | 2-3 | 2 |
| | | DQB1*0202, 0501 | | 24 |
| | | | | 30 |
| | | | | 34 |
| 22 | HSV1+/2− | DRB1*04011, 04011, | 24-3 | 2 |
| | | DQB1*0301, 0301 | | 24 |
| | | | | 30 |
| | | | | 34 |
| 23 | HSV1+/2− | DRB1*07011, 15011, | 2-5, 2-2, 2-1, 2-4, 2-7, 2 | 2 |
| | | DQB1*0303, 0602 | 24-1, 24-3, 24-7 | 24 |
| | | | | 30 |
| | | | 34-6 | 34 |
| 24 | HSV1+/2 | DRB1*01011, 04051, | 2-2 | 2 |
| | | DQB1*0202, 0501 | 24-3, 24-4 | 24 |
| | | | 30-4, 30-1 | 30 |
| | | | | 34 |

1. Peptide 1 = AAs 1-20, Peptide 2 = AAs 11-30, Peptide 3 = AAs 21-40 etc.
2. Peptide x > peptide y indicates peptide x was more immunostimulatory than peptide y.
3. Peptide x′ = peptide y′ indicates peptide x′ exhibited the same immunogenicity as peptide y′.
*34-1 refers to the 1^{st} of the nine 12 mers within 20 mer peptide 34.

### Definition of gD2 Peptide Epitopes by Human CD4 Lymphocytes

The entire sequence of gD from the HSV type 2 (clinical isolate #356.2038 strain) and the key peptide 20 mer epitopes identified in this study are shown in FIG. 1.

Reactivity of CD4 lymphocytes from HSV2 seropositive subjects with a recurrent history of a recurrent genital herpes was tested against the full panel of 20 mer peptides using the ⁵¹ Cr release cytotoxicity assay and confirmed by IFN-γ ELISpot assays. Initially autologous LCL from 12 patients with recent genital herpes were sensitised with each peptide of the entire HSV2 gD library and tested against patients CD4 lymphocytes in a bulk cytotoxicity assay at E:T ratios of 5:1 and 25:1. Patient CD4 lymphocytes were tested against each peptide individually but, for logistical reasons, usually in two overlapping sets of 20 or 19 consecutive 20 mer peptides as odd or even numbered peptides: 1, 3, ---, to 33 or 2, 4, ---, to 34 per bleed for each patient.

Experimental results for E:T of 5:1 and 25:1 were found not to be significantly different (p>0.5) from each other (data not shown), so only the results for selected experiments with an E:T of 5:1 are presented. In the 12 patients initially tested (shown in Table 3A) and another four (in Table 3B) there was CD4 lymphocyte recognition of at least some gD2 peptides varying from two to six of the 20 peptides tested in one batch. The peptides 2, 24, 30 and 34 were the most frequently recognised even numbered peptides followed by peptides 10, 12 and 26 (see FIG. 2 and Table 3A) and this was confirmed by IFN-γ ELISpot. Where one of these even numbered peptides was recognised, either or both flanking odd numbered peptides were also usually recognised e.g. peptides 1 and 3 for peptide 2 or peptides 33 and 35 for peptide 34 (Table 3).

Definition of Peptide Epitopes within the Native Viral Protein gD2 and their MHC II Restriction

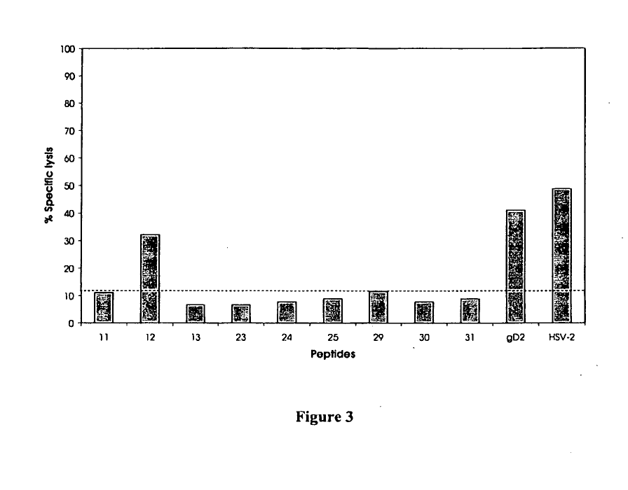

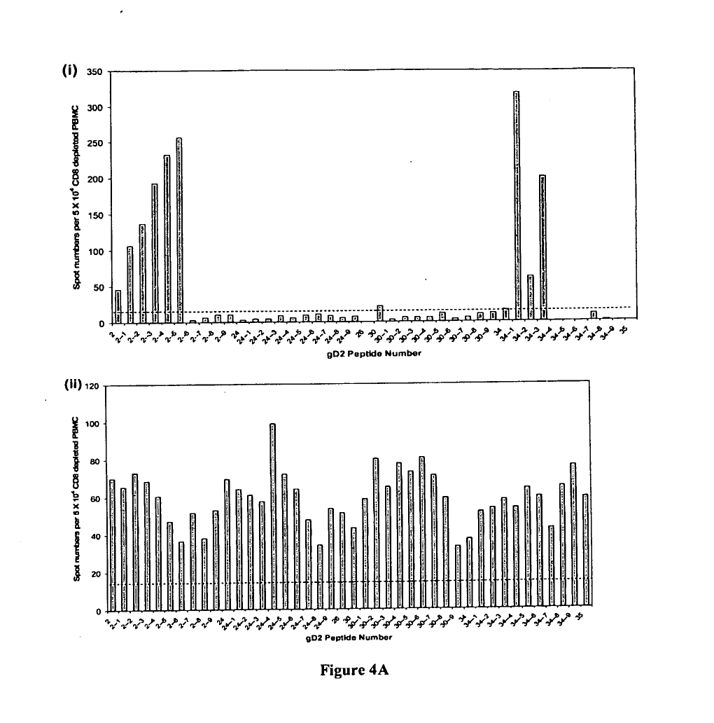

To confirm that these peptides truly contained epitopes recognised by gD2 specific CD4 lymphocytes, autologous effector T-cell lines from three HSV2 seropositive patients were restimulated in vitro with peptides 2 or 12 and the effectors were tested initially by ⁵¹Cr release assay, against peptide sensitised target autologous PHA blasts and, as controls, target cells infected with HSV2, and recombinant gD2 incubated targets. These CD4 effector T cells when stimulated by peptides 2 or 12 showed high levels of specific activity only against either peptide 2 or 12 sensitised target cells respectively and, in each case, also against gD2 sensitised and HSV2 infected target cells. FIG. 3 shows a representative experiment for peptide 12.

MHC II specificity and its nature were determined by incubation of peptide sensitised target cells with anti-HLA-DR and DQ antibodies. As shown in Table 4 CD4 lymphocyte recognition of peptides 1 and 33 was ablated by anti-HLA-DR antibodies. Peptide 24-4 and 30-5 appeared to be restricted only to HLA-DR (Table 4). However with other peptides such as peptide 30-5 in some patients these results were not so distinct, with predominant inhibition by anti-HLA-DR and also partial inhibition by anti-HLA-DQ antibodies (Table 4). MHC II typing of all patients and the predominant peptides recognised by these patients are shown in Table 3.

TABLE 4
HLA-DR/DQ specificity of HSV2 peptide
recognition by HSV infected patients

| | % inhibition of CD4 lymphocyte response to peptides | | |
|---|---|---|---|
| | measured as ⁵¹Cr release assay or IFN-γ ELISA | | |
| Peptides | No blocking | Anti-HLA-DR | Anti-DQ |

20 mers

| | | | |
|---|---|---|---|
| 1 | 0 | 88 ± 6% | −8 ± 6% |
| 33 | 0 | 90 ± 7% | +10 ± 6% |
| 35 | 0 | 77 ± 6% | −11 ± 7% |

Controls

| | | | |
|---|---|---|---|
| gD2 | 0 | 38 ± 5% | +15 ± 5% |
| HSV2 | 0 | 33 ± 4% | +12 ± 4% |

12 mers

| | | | |
|---|---|---|---|
| 24-4⁺⁺ | 0 | 64 ± 16% | 0 |
| 30-5⁺⁺ | 0 | 73 +/− 11%% | 53.8 +/− 3.8% |

LCL were pre-incubated with either anti-HLA-DR, anti HLA-DQ (BD Pharmingen, San Diego, CA) or isotype control antibodies for 30 mins at a final concentration of 50 μg/ml prior to peptide pulse for 2 hrs. 20 mer peptides 1, 33, 35 previously recognised as targets, control recombinant gD2, and HSV-2 were incubated with the CD4 T lymphocytes of patient 4 and then tested in ⁵¹Cr release assay. 12 mer peptides 24-4 and 30-5 were incubated with CD4 lymphocytes of patient 20 and 19, respectively after pre-treatment of the cells with anti-HLA-DR and DQ antibodies. Supernatants were tested for interferon-γ by ELISA (Endogen, Rockford, IL). IFN-γ ELISA was performed according to the manufacturer's instruction. Experiments were performed in triplicate and results expressed as mean ± standard deviation.

Fine mapping of the minimal epitopes within peptides 2, 24, 30, and 34

Serial 12 mers within the 20 mers 2, 24, 30 and 34, selected as the most frequent immunodominant epitopes were used for fine mapping of CD4 T lymphocyte epitopes.

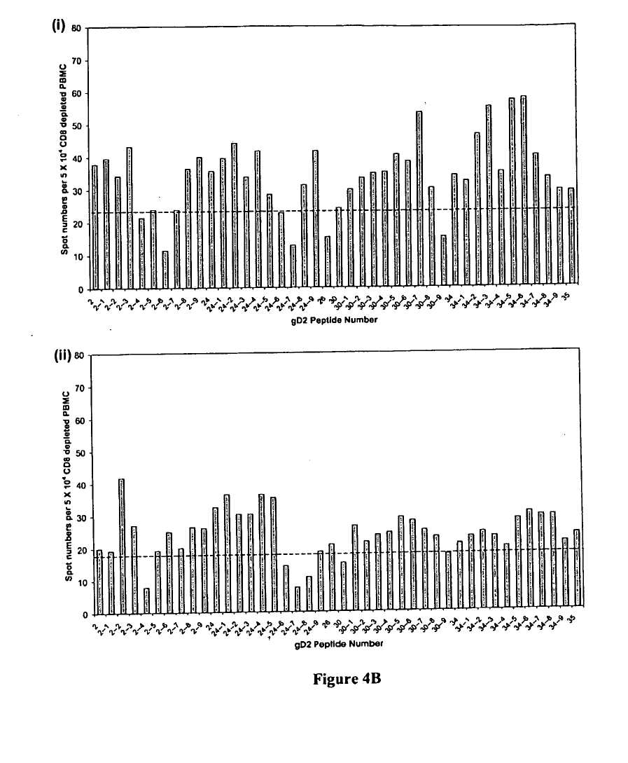

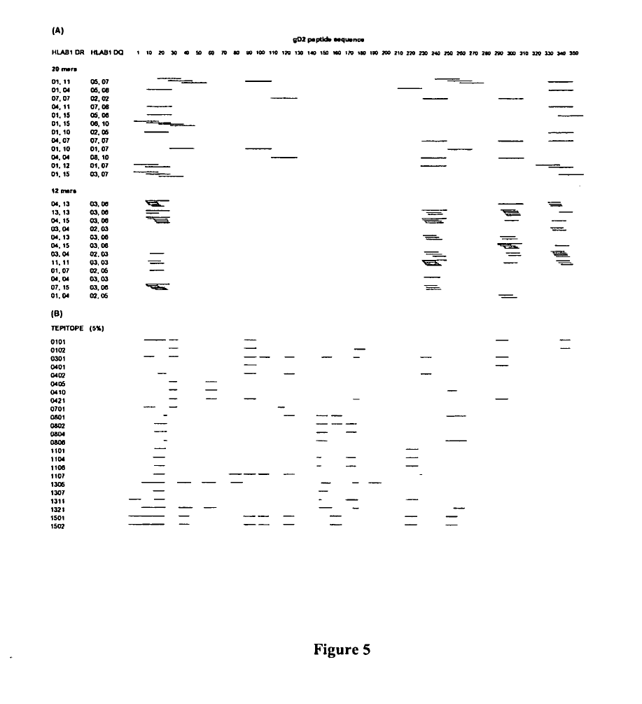

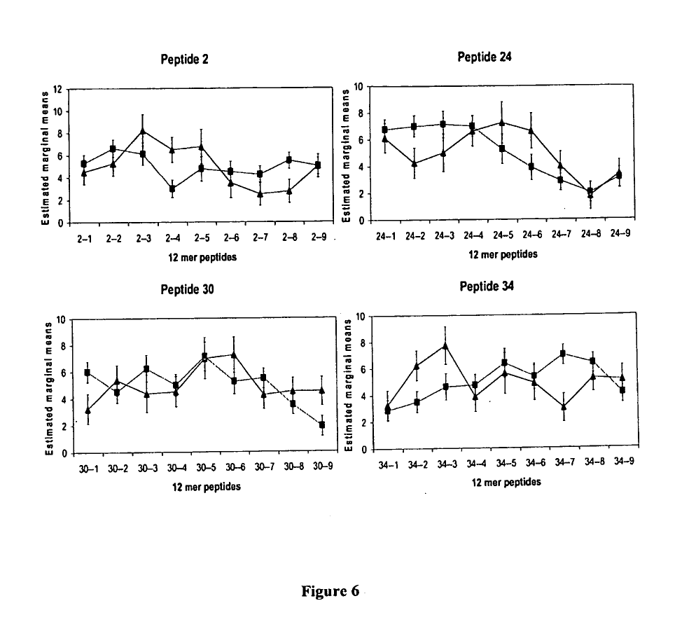

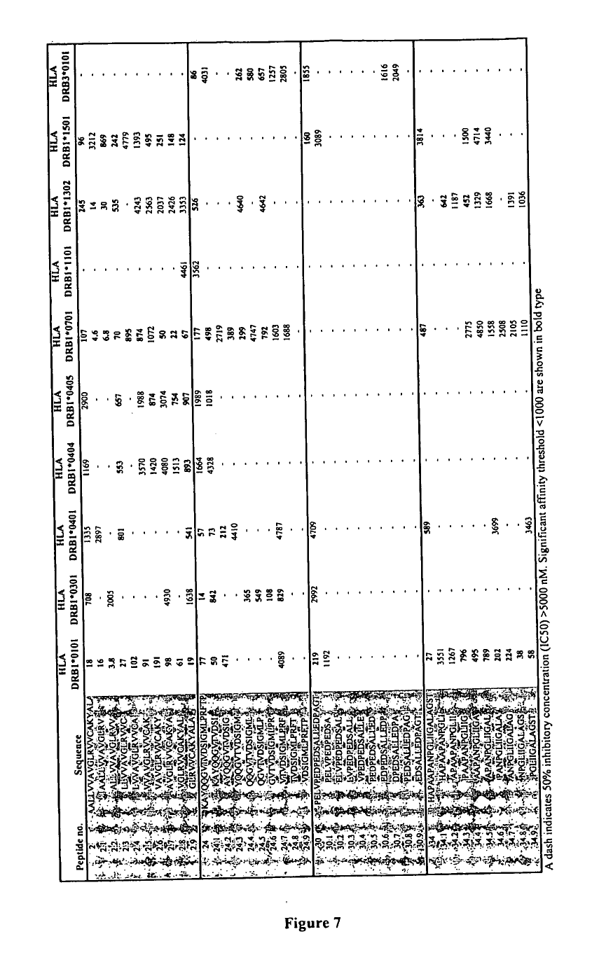

In some patients definition of minimal epitopes was clear, as shown in FIG. 4A where there is an increasing recognition of peptides 2.1 to 2.4 and no recognition thereafter within peptide 2 (patient 13, FIG. 4A (i)). However in other patients it was very difficult to define minimal epitopes within the 20 mers (patient 14, FIG. 4A (ii)). Furthermore, as for the 20 mers it was impossible to assign recognition of individual 12 mers to specific MHC II (HLA-DR) alleles. Indeed, comparisons of the patterns of recognition between patients of different HLA-DR and DQ types indicate cross recognition of specific 12 mer and 20 mer peptide epitopes across different MHC alleles.

Comparison of Empirically Defined Epitopes with Those Predicted by the TEPITOPE Algorithm

According to the TEPITOPE algorithm there was a very high density of predicted epitopes in gD2 for the commonest MHC II alleles, whether the predictive levels were set at 2% or 5%, e.g. 4 epitopes recognised by at least 10 alleles were predicted within peptide 2 when set at the 5% level (FIG. 5). The algorithm also predicted cross recognition and binding of different epitopes within 12 mer or 20 mer peptides according to different MHC II alleles (see Bian and Hammer, 2004, Discovery of promiscuous HLA-II-restricted T cell epitopes with TEPITOPE, Methods 34:468; Bian et al., 2003, The use of bioinformatics for identifying class II-restricted T-cell epitopes., Methods 29:299), e.g. peptide epitopes within the 20 mer peptide 2 for HLA DRB1*0101, 0301, 0402, 0701, 1101/1104/1106, 1305, and 1501/1502. In some cases there was a good correlation between predicted and empirically determined epitopes (e.g. within peptides 2 and 10) whereas in others the correlation was poor (e.g. peptides 24 and 34 (FIG. 5). This promiscuous cross recognition and density of predicted epitopes was consistent with the in vitro binding data (FIG. 7) and the CD4 T cell responses by IFN-γ ELISpot (FIG. 5).

The high density of predicted epitopes within gD was also reflected in the number of epitopes predicted to be recognised per HLA-DR or DQ type e.g. 3-8 peptides per type at the 5% level. Furthermore, the algorithm predicted promiscuous recognition of peptide epitopes across several MHC II types e.g. peptide epitopes within the 20 mer peptide 2 for HLA-DR 1, 4. This density of predicted epitopes was consistent with our difficulties in defining minimal epitopes even for individual patients who were homozygous for HLA-DR 11 and 13 (patient 20 and 14, respectively).

### Cross Reactive Recognition of HSV2 Peptides by CD4 Lymphocytes for HSV1 Seropositive Subjects

Several of the peptides were cross recognised by patients who were HSV1+/2− (FIG. 4B). In some cases their CD4 lymphocytes responded to these peptides at lower levels than that by patients who were HSV1−/2+ whereas in other cases CD4 lymphocyte responses were as strong. The comparison of the four key selected 20 mer peptides and their serial overlapping 12 mers for cross-reactivity between HSV2 and HSV1 is shown in FIG. 6 all were recognised by HSV1+/2− patients. Because of their promiscuous recognition across different MHC II alleles it was possible to conduct a comparative is quantitative study of recognition of the nine internal serial 12 mers for each 20 mer, peptides 2, 24, 30, 34 by CD4 lymphocytes from eight HSV1+/2- and four HSV1−/2+ patients.

As shown in FIG. 6 the patterns of recognition were very similar between CD4 lymphocytes of HSV1+ and 2+ patients for peptide 30 and mostly similar for peptides 2, 24, and 34. Peptide 34 showed the greatest differences in predominant recognition of internal peptides at the N and C-terminal regions for HSV1+ and HSV2+ patients. For peptides 2 and 24 recognition of the 12 mers in the amino half of the 20 mer was dominant.

Peptides 30 and 24 have the greatest degree of homology between the HSV1 and 2 sequences (FIG. 1). The slight differences in responses to some of the 12 mers in peptides 2 and 34 may be explained by unlike amino acid substitutions at positions 4, 14, for peptide 2 and positions 3, 6, 8, 10 in peptide 34 especially prolines at positions 3 and 10.

No statistically significant interaction between the effects of internal peptide number and HSV status was found for peptide 30 (p=0.239). Nor were there any major amino acid differences between HSV1 and HSV2 in this 20 mer. As was therefore anticipated, none of the Mann-Whitney tests of the rank scores by HSV group were statistically significant for any of the 9 internal peptides.

However, peptides 2, 24 and 34 demonstrated a statistically significant interaction between the effects of internal peptide number and HSV1/2 status (p-value for interaction 0.098, 0.057, 0.068 respectively). For peptide 2, there were major amino acid differences between internal peptide 4 and, to a lesser extent, for internal peptide 3, of HSV1 and HSV2. Mann-Whitney tests of the rank scores by HSV1/2 status showed a statistically significant difference in recognition by HSV1 and 2 patients for internal peptide 4 (p=0.048) but not for internal peptide 3 (p=0.461).

For peptide 24, there was no amino acid difference between internal peptides 2 to 8 of HSV1 and 2. Nevertheless Mann-Whitney tests of the rank scores by HSV1/2 status showed a statistically significant difference for internal peptides 2 (p=0.048) and 6 (p=0.048) but not for the other internal peptides.

For peptide 34, there were significant amino acid differences between internal peptides 2, 3 of HSV1 and 2 whilst a proline was present at the second AA of internal peptide 7. Mann-Whitney tests of the rank scores by HSV group showed statistically significant differences for all of these internal peptides 2 (p=0.048), 3 (p=0.028) and 7 (p=0.016).

In summary, for the two 20 mer peptides in which major amino acid differences between HSV1 and HSV2 were identified for some internal peptides, there was a statistically significant interaction between the effects of internal peptide number (1 to 9) and HSV status (HSV1 only or HSV2 only) on the rank scores. Considering the total number of internal peptides (36), for the nine in which major amino acid differences were identified, statistically significant differences in 12 mer recognition according to HSV1/2 were detected in four. Among the 27 remaining internal peptides in which no major amino acid difference was identified, there were only two internal peptides with a statistically significant difference between the rank scores by HSV1/2 status (internal peptides 2 and 6 for peptide 24). Thus HSV1/2 sequence comparisons correlated with observed 12 mer peptide recognition (4/9 vs 2/27, p=0.014, Fishers Exact Test), providing validation for the experimental results.

### Binding of Peptides to HLA DR Molecules

The set of epitopes identified were assessed to determine whether they showed promiscuous HLA DR binding affinity. Each of the 20 mer epitopes was tested for its capacity to bind to a panel of 10 common DR molecules. As shown in FIG. 7, peptides 2 and 24 were found to be degenerate binders, binding 50% or more of the molecules tested with high affinity (IC₅₀<1000 nM). These epitopes also bound several other specificities with intermediate affinity (IC₅₀ 1000-5000 nM). Peptide 34 was less degenerate, but still bound 4 of the 10 DR molecules tested with high affinity. By contrast, peptide 30 bound only DRB1*0101 and DRB1*1501. The peptide 35 20 mer which was less immunogenic than other four also bound to DRB1*0101, 0404, 0701, and 1302.

Analysis of the pattern of binding of the corresponding 12 mer truncations of each epitope was also undertaken. The data, shown in FIG. 7, revealed in several cases that binding of the 20 mer peptide may be achieved by use of more than one core region. For example, in the case of peptide 2 binding to DRB1*0101, DRB1*0701 and DRB1*1501 was observed with truncations incorporating either the N- or C-terminal regions of the peptide. In the majority of other cases optimal binding appeared to be more concentrated in one region of the 20 mer peptide. However, because the binding groove of the DR molecule allows for multiple core alignments even in the context of a 12 mer peptide, the possibility that each of these regions represents multiple closely nested epitopes cannot be excluded.

Taken together with the antibody blocking data, these binding data support DR restriction in the majority of cases for peptides 2, 24 and 34, and suggest that binding in several contexts may involve more than one core region. These data also suggest that, at least in some cases, peptide 30 may be DQ restricted. In most cases there was a good correlation between peptide binding to HLA-DR and T cell responses to the peptides in subjects of similar HLA_DR types (e.g. 8 of 10 HLA DRB1*0101 positive subjects recognise epitopes in peptide 2.

### Discussion

In this work CD4 lymphocytes of all HSV2 positive patients with genital herpes were demonstrated to recognise at least two to six of an overlapping library of peptide 20 mers spanning the whole glycoprotein gD2 sequence, including the leader sequence. Bulk CD4 lymphocytes were used to identify specific epitopes in an effort to minimise the bias induced by using T-cell clones. However this did reduce the magnitude of response, requiring the use of interferon gamma Elispot or ⁵¹Cr release assay for maximum sensitivity. This is consistent with the relatively low frequency of 0.2%-0.4% of gD peptide specific CD4 lymphocytes in vivo shown by intracellular cytokine staining for interferon-γ (Gonzalez et al., 2005, Expression of cutaneous lymphocyte-associated antigen and E-selectin ligand by circulating human memory CD4 T lymphocytes specific for herpes simplex virus type 2, J. Infect. Dis. 191:243; Asanuma et al., 2000, Frequencies of memory T cells specific for varicella-zoster virus, herpes simplex virus, and cytomegalovirus by intracellular detection of cytokine expression, J Infect Dis 181: 859). In some cases CD4 lymphocyte responses were observed in patients soon after recurrence of genital herpes but declined to undetectable when re-tested six months later (unpublished observations). This low frequency of responder in HSV seropositive patients contrasts with those in the other herpesvirus infections, cytomegalovirus (1.2%) and EBV (Asanuma et al., 2000, Frequencies of memory T cells specific for varicella-zoster virus, herpes simplex virus, and cytomegalovirus by intracellular detection of cytokine expression, J Infect Dis 181: 859). Identification of key 20 mer peptide epitopes (shown in Table 3A) was confirmed by both ⁵¹Cr release assays using LCLs as targets and ELISpot assays and the specificity of gD2 peptide specific T cell lines were verified by ⁵¹Cr release assay using PHA blasts as target cells to reduce non-specific lysis.

Serially truncated 12 mers within the initially identified 20 mer peptide epitope were used to define the minimal T-cell epitopes in most patients, but in others this was impossible suggesting clustering or overlapping of more than one epitope in the 20 mer. It was also difficult to assign minimal 12 mer peptides to an individual HLA-DR or DQ allele. Nevertheless in all four patients tested the peptide epitopes were HLA-DR restricted but most peptides were recognised in the context of several HLA-DR alleles. This was facilitated by the homozygous HLA-DR state in some of the patients. In some patients anti-HLA-DR antibodies did not completely inhibit recognition of gD2 and anti-HLA-DQ was also partially inhibitory, suggesting there might still be some HLA-DQ specific peptides within gD2. The TEPITOPE program which predicts MHC H specific peptide epitopes within a protein suggested possible reasons for the difficulty in defining individual 20 mer epitopes in some patients (Bian and Hammer, 2003, The use of bioinformatics for identifying class II-restricted T-cell epitopes, Methods 29:299). At the most stringent levels several overlapping or clustered MHC II restricted epitopes were predicted to occur (within 20 mers) at various places within the gD2 molecule. The cross recognition of individual 20 mer epitopes by patients of completely different MHC II types is consistent with promiscuous cross recognition of individual minimal 12 mer epitopes by the individual MHC II molecules themselves or alternatively in some cases, co-expression of two epitopes within the same peptide which are then recognised by different MHC types. Nevertheless as shown in FIG. 5 there was not a complete correlation between our empirical results obtained by screening and those epitopes predicted by TEPITOPE e.g. the consistent recognition of epitopes in 330-350 region by patients with a broad range of HLA-DR alleles was not predicted.

Cross recognition of each of four major 20 mer peptide epitopes (peptides 2, 24, 30, 34) and of the nine serial overlapping minimal T-cell epitopes within each 20 mer was examined in patients who were seropositive only for HSV1 or for HSV2. All four 20 mer peptides were cross recognised. Because of the promiscuity of recognition of the serial internal 12 mer epitopes across different HLA-DR alleles, consensus recognition patterns amongst eight HSV 1 seropositive and four HSV2 seropositive patients became apparent. For one of these 20 mers (p30) the pattern of recognition along the 20 mer was very similar by HSV1 or 2 seropositive patients, reflecting the sequence homology in those regions of glycoproteins D2 and D1. In another two (peptides 2 and 34) there was similarity in recognition in one but not in another region of the 20 mer. These differences in the two 20 mers were correlated with more marked differences in HSV1/2 sequence homology (FIG. 1). In peptide 24 there was a slight difference in recognition patterns for HSV1 and 2 but this was not obvious from inspection of sequences. Differences in binding of the 12 mer peptide to the key binding pockets of different HLA alleles or to the TCR might account for such differences. Nevertheless overall relatively simple statistical modelling suggested a high degree of correlation between sequences predicted and observed CD4 lymphocyte responses.

The presence of multiple epitopes scattered throughout the gD epitope and which can be cross recognised by humans or mice of different MHC types was demonstrated. Importantly these epitopes induced Th1 rather than the Th2 responses of some other gD epitopes and when the Th1 epitopes were used to immunize mice they protected against viral challenge. The major gD epitopes in our study were detected by cytotoxicity and interferon gamma ELISpot assays indicating they were also Th1 epitopes and may be useful as immunogens in future.

The whole of the gD2 molecule was examined, including the leader sequence (resulting in a 25AA shift in numbering systems between the studies) and the trans-membrane region of gD2. We reasoned that gD2 was clinically a more important molecule and secondly that the leader and transmembrane sequences might still be important in generating CD4 lymphocyte responses, especially following uptake of apoptotic or necrotic debris by antigen presenting cells (although usually inducing an MHC I restricted response). In support of this hypothesis the TEPITOPE algorithm indicates the presence of potential broadly recognised MHC II epitopes in the region 10-30 of the immature gD molecule. Furthermore, the serial internal 12 mer peptide studies showed recognition by HSV1 seropositive patients in regions of peptide 2 Peptide 34 (AAs 331-350) in this study includes the transmembrane region which showed the greatest difference in recognition between HSV1+ and HSV2+ patients with clear separation of epitopes in the amino and carboxy terminal region.

The detection of broad human responses (across HLA-DR alleles) to peptides 24 (AAs 231-250) and 30 (AAs 291-310) in this study probably reflects the enhanced sensitivity of ELISpot assay over T-lymphocyte proliferation for CD4 lymphocyte responses. These responses were confirmed with the serial 12 mer studies which also showed p34 to be the most divergent in recognition by HSV1 and HSV2 seropositive patients of the four key peptides epitopes.

1.-54. (canceled)

55. An isolated immunogenic Herpes simplex virus (HSV) glycoprotein D peptide consisting of 23 amino acids or less, said peptide comprising an amino acid sequence selected from the group consisting of SEQ ID Nos: 1-5, SEQ ID Nos: 9-12, SEQ ID Nos: 23-27, SEQ ID Nos: 29-31 and SEQ ID Nos: 33-37, or an immunogenic fragment or variant thereof.

56. The peptide according to claim 55 wherein said immunogenic fragment or variant comprises an amino acid selected from the group consisting of SEQ ID Nos: 46-71.

57. The peptide according to claim 55 wherein the peptide is a component portion of a fusion protein or polypeptide.

58. The peptide according to claim 57 wherein the fusion protein comprises a plurality of isolated immunogenic HSV glycoprotein D peptides.

59. The peptide according to claim 57 wherein the fusion protein or polypeptide comprises a polypeptide sequence unrelated to the immunogenic HSV glycoprotein D peptides.

60. The peptide according to claim 55 wherein the HSV glycoprotein D peptide is an HSV glycoprotein D2 peptide.

61. A polynucleotide sequence comprising a nucleic acid sequence encoding a peptide, immunogenic fragment or variant according to claim 55.

62. A method for inducing an immune response to HSV in a subject, the method comprising administering to said subject an effective amount of at least one immunogenic HSV glycoprotein D peptide consisting of 23 amino acids or less, said peptide comprising an amino acid sequence selected from the group consisting of SEQ ID Nos 1-5, SEQ ID Nos: 9-12, SEQ ID Nos: 23-27, SEQ ID Nos: 29-31 and SEQ ID Nos: 33-37, or an immunogenic fragment or variant thereof.

63. The method according to claim 62 wherein said inducing is for treatment of an HSV infection in a subject.

64. The method according to claim 62 wherein said inducing is for prevention of an HSV infection in a subject.

65. The method according to claim 62 wherein the HSV is HSV1 and HSV2.

66. A method for determining the level of T-lymphocyte immunity to HSV1 or HSV2 in a patient, comprising:
(a) obtaining a biological sample from the patient;
(b) contacting the sample with a peptide consisting of 23 amino acids or less, said peptide comprising an amino acid sequence selected from the group consisting of SEQ ID Nos: 1-5, SEQ ID Nos: 9-12, SEQ ID Nos: 23-27, SEQ ID Nos: 29-31 and SEQ ID Nos: 33-37, or an immunogenic fragment or variant thereof; and
(c) quantifying T-lymphocytes that respond to the peptide.

67. The method according to claim 62 wherein said immunogenic fragment or variant comprises an amino acid sequence selected from the group consisting of SEQ ID Nos: 41-76.

68. The method according to claim 62 wherein the HSV glycoprotein D peptide is an HSV glycoprotein D2 peptide.

69. A diagnostic or prognostic kit comprising at least one component selected from the group consisting of:
(a) an isolated immunogenic Herpes simplex virus (HSV) glycoprotein D peptide consisting of 23 amino acids or less, said peptide comprising an amino acid sequence selected from the group consisting of SEQ ID Nos: 1-5, SEQ ID Nos: 9-12, SEQ ID Nos: 23-27, SEQ ID Nos: 29-31 and SEQ ID Nos: 33-37, or an immunogenic fragment or variant thereof;
(b) a polypeptide comprising at least one peptide according to part (a);
(c) a plurality of peptides according to part (a); and
(d) a tetramer reagent comprising a fragment of an HLA-DR molecule bound to a peptide according to part (a).

70. The diagnostic or prognostic kit according to claim 69 wherein said immunogenic fragment or variant comprises an amino acid selected from the group consisting of SEQ ID Nos: 46-71.

71. The diagnostic or prognostic kit according to claim 69 wherein the HSV glycoprotein D peptide is an HSV glycoprotein D2 peptide.

72. The method according to claim 66 wherein said immunogenic fragment or variant comprises an amino acid sequence selected from the group consisting of SEQ ID Nos: 41-76.


SEQUENCE LISTING (76 sequences)

SEQ ID NO: 1 - protein, 20 aa, Herpes Simplex Virus 2 Strain 186
MGRLTSGVGTAALLVVAVGL

SEQ ID NO: 2 - protein, 20 aa, Herpes Simplex Virus 2 Strain 186
AALLVVAVGLRVVCAKYALA

SEQ ID NO: 3 - protein, 20 aa, Herpes Simplex Virus 2 Strain 186
RVVCAKYALADPSLKMADPN

SEQ ID NO: 4 - protein, 20 aa, Herpes Simplex Virus 2 Strain 186
DPSLKMADPNRFRGKNLPVL

SEQ ID NO: 5 - protein, 20 aa, Herpes Simplex Virus 2 Strain 186
RFRGKNLPVLDQLTDPPGVK

SEQ ID NO: 6 - protein, 20 aa, Herpes Simplex Virus 2 Strain 186
DQLTDPPGVKRVYHIQPSLE

SEQ ID NO: 7 - protein, 20 aa, Herpes Simplex Virus 2 Strain 186
RVYHIQPSLEDPFQPPSIPI

SEQ ID NO: 8 - protein, 20 aa, Herpes Simplex Virus 2 Strain 186
DPFQPPSIPITVYYAVLERA

SEQ ID NO: 9 - protein, 20 aa, Herpes Simplex Virus 2 Strain 186
TVYYAVLERACRSVLLHAPS

SEQ ID NO: 10 - protein, 20 aa, Herpes Simplex Virus 2 Strain 186
CRSVLLHAPSEAPQIVRGAS

SEQ ID NO: 11 - protein, 20 aa, Herpes Simplex Virus 2 Strain 186
EAPQIVRGASDEARKHTYNL

SEQ ID NO: 12 - protein, 20 aa, Herpes Simplex Virus 2 Strain 186
DEARKHTYNLTIAWYRMGDN

SEQ ID NO: 13 - protein, 20 aa, Herpes Simplex Virus 2 Strain 186
TIAWYRMGDNCAIPITVMEY

SEQ ID NO: 14 - protein, 20 aa, Herpes Simplex Virus 2 Strain 186
CAIPITVMEYTECPYNKSLG

SEQ ID NO: 15 - protein, 20 aa, Herpes Simplex Virus 2 Strain 186
TECPYNKSLGVCPIRTQPRW

SEQ ID NO: 16 - protein, 20 aa, Herpes Simplex Virus 2 Strain 186
VCPIRTQPRWSYYDSFSAVS

SEQ ID NO: 17 - protein, 20 aa, Herpes Simplex Virus 2 Strain 186
SYYDSFSAVSEDNLGFLMHA

SEQ ID NO: 18 - protein, 20 aa, Herpes Simplex Virus 2 Strain 186
EDNLGFLMHAPAFETAGTYL

SEQ ID NO: 19 - protein, 20 aa, Herpes Simplex Virus 2 Strain 186
PAFETAGTYLRLVKINDWTE

SEQ ID NO: 20 - protein, 20 aa, Herpes Simplex Virus 2 Strain 186
RLVKINDWTEITQFILEHRA

SEQ ID NO: 21 - protein, 20 aa, Herpes Simplex Virus 2 Strain 186
ITQFILEHRARASCKYALPL

SEQ ID NO: 22 - protein, 20 aa, Herpes Simplex Virus 2 Strain 186
RASCKYALPLRIPPAACLTS

SEQ ID NO: 23 - protein, 20 aa, Herpes Simplex Virus 2 Strain 186
RIPPAACLTSKAYQQGVTVD

SEQ ID NO: 24 - protein, 20 aa, Herpes Simplex Virus 2 Strain 186
KAYQQGVTVDSIGMLPRFTP

SEQ ID NO: 25 - protein, 20 aa, Herpes Simplex Virus 2 Strain 186
SIGMLPRFTPENQRTVALYS

SEQ ID NO: 26 - protein, 20 aa, Herpes Simplex Virus 2 Strain 186
ENQRTVALYSLKIAGWHGPK

SEQ ID NO: 27 - protein, 20 aa, Herpes Simplex Virus 2 Strain 186
LKIAGWHGPKPPYTSTLLPP

SEQ ID NO: 28 - protein, 20 aa, Herpes Simplex Virus 2 Strain 186
PPYTSTLLPPELSDTTNATQ

SEQ ID NO: 29 - protein, 20 aa, Herpes Simplex Virus 2 Strain 186
ELSDTTNATQPELVPEDPED

SEQ ID NO: 30 - protein, 20 aa, Herpes Simplex Virus 2 Strain 186
PELVPEDPEDSALLEDPAGT

SEQ ID NO: 31 - protein, 20 aa, Herpes Simplex Virus 2 Strain 186
SALLEDPAGTVSSQIPPNWH

SEQ ID NO: 32 - protein, 20 aa, Herpes Simplex Virus 2 Strain 186
VSSQIPPNWHIPSIQDVAPH

SEQ ID NO: 33 - protein, 20 aa, Herpes Simplex Virus 2 Strain 186
IPSIQDVAPHHAPAAPANPG

SEQ ID NO: 34 - protein, 20 aa, Herpes Simplex Virus 2 Strain 186
HAPAAPANPGLIIGALAGST

SEQ ID NO: 35 - protein, 20 aa, Herpes Simplex Virus 2 Strain 186
LIIGALAGSTLAALVIGGIA

SEQ ID NO: 36 - protein, 20 aa, Herpes Simplex Virus 2 Strain 186
LAALVIGGIAFWVRRRRSVA

SEQ ID NO: 37 - protein, 20 aa, Herpes Simplex Virus 2 Strain 186
FWVRRRRSVAPKRLRLPHIR

SEQ ID NO: 38 - protein, 20 aa, Herpes Simplex Virus 2 Strain 186
PKRLRLPHIRDDDAPPSHQP

SEQ ID NO: 39 - protein, 16 aa, Herpes Simplex Virus 2 Strain 186
DDDAPPSHQPLFYPRE

SEQ ID NO: 40 - protein, 396 aa, Herpes Simplex Virus 2 Strain 186
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFTPENQRTVALYSLKIAGWHGPKPPYTSTLLPPELSDTTNATQPELVPEDPEDSALLEDPAGTVSSQIPPNWHIPSIQDVAPHHAPAAPANPGLIIGALAGSTLAALVIGGIAFWVRRRRSVAPKRLRLPHIRDDDAPPSHQPLFYPRE

SEQ ID NO: 41 - protein, 12 aa, Herpes Simplex Virus 2 Strain 186
AALLVVAVGLRV

SEQ ID NO: 42 - protein, 12 aa, Herpes Simplex Virus 2 Strain 186
ALLVVAVGLRVV

SEQ ID NO: 43 - protein, 12 aa, Herpes Simplex Virus 2 Strain 186
LLVVAVGLRVVC

SEQ ID NO: 44 - protein, 12 aa, Herpes Simplex Virus 2 Strain 186
LVVAVGLRVVCA

SEQ ID NO: 45 - protein, 12 aa, Herpes Simplex Virus 2 Strain 186
VVAVGLRVVCAK

SEQ ID NO: 46 - protein, 12 aa, Herpes Simplex Virus 2 Strain 186
VAVGLRVVCAKY

SEQ ID NO: 47 - protein, 12 aa, Herpes Simplex Virus 2 Strain 186
AVGLRVVCAKYA

SEQ ID NO: 48 - protein, 12 aa, Herpes Simplex Virus 2 Strain 186
VGLRVVCAKYAL

SEQ ID NO: 49 - protein, 12 aa, Herpes Simplex Virus 2 Strain 186
GLRVVCAKYALA

SEQ ID NO: 50 - protein, 12 aa, Herpes Simplex Virus 2 Strain 186
KAYQQGVTVDSI

SEQ ID NO: 51 - protein, 12 aa, Herpes Simplex Virus 2 Strain 186
AYQQGVTVDSIG

SEQ ID NO: 52 - protein, 12 aa, Herpes Simplex Virus 2 Strain 186
YQQGVTVDSIGM

SEQ ID NO: 53 - protein, 12 aa, Herpes Simplex Virus 2 Strain 186
QQGVTVDSIGML

SEQ ID NO: 54 - protein, 12 aa, Herpes Simplex Virus 2 Strain 186
QGVTVDSIGMLP

SEQ ID NO: 55 - protein, 12 aa, Herpes Simplex Virus 2 Strain 186
GVTVDSIGMLPR

SEQ ID NO: 56 - protein, 12 aa, Herpes Simplex Virus 2 Strain 186
VTVDSIGMLPRF

SEQ ID NO: 57 - protein, 12 aa, Herpes Simplex Virus 2 Strain 186
TVDSIGMLPRFT

SEQ ID NO: 58 - protein, 12 aa, Herpes Simplex Virus 2 Strain 186
VDSIGMLPRFTP

SEQ ID NO: 59 - protein, 12 aa, Herpes Simplex Virus 2 Strain 186
PELVPEDPEDSA

SEQ ID NO: 60 - protein, 12 aa, Herpes Simplex Virus 2 Strain 186
ELVPEDPEDSAL

SEQ ID NO: 61 - protein, 12 aa, Herpes Simplex Virus 2 Strain 186
LVPEDPEDSALL

SEQ ID NO: 62 - protein, 12 aa, Herpes Simplex Virus 2 Strain 186
VPEDPEDSALLE

SEQ ID NO: 63 - protein, 12 aa, Herpes Simplex Virus 2 Strain 186
PEDPEDSALLED

SEQ ID NO: 64 - protein, 12 aa, Herpes Simplex Virus 2 Strain 186
EDPEDSALLEDP

SEQ ID NO: 65 - protein, 12 aa, Herpes Simplex Virus 2 Strain 186
DPEDSALLEDPA

SEQ ID NO: 66 - protein, 12 aa, Herpes Simplex Virus 2 Strain 186
PEDSALLEDPAG

SEQ ID NO: 67 - protein, 12 aa, Herpes Simplex Virus 2 Strain 186
EDSALLEDPAGT

SEQ ID NO: 68 - protein, 12 aa, Herpes Simplex Virus 2 Strain 186
HAPAAPANPGLI

SEQ ID NO: 69 - protein, 12 aa, Herpes Simplex Virus 2 Strain 186
APAAPANPGLII

SEQ ID NO: 70 - protein, 12 aa, Herpes Simplex Virus 2 Strain 186
PAAPANPGLIIG

SEQ ID NO: 71 - protein, 12 aa, Herpes Simplex Virus 2 Strain 186
AAPANPGLIIGA

SEQ ID NO: 72 - protein, 12 aa, Herpes Simplex Virus 2 Strain 186
APANPGLIIGAL

SEQ ID NO: 73 - protein, 12 aa, Herpes Simplex Virus 2 Strain 186
PANPGLIIGALA

SEQ ID NO: 74 - protein, 12 aa, Herpes Simplex Virus 2 Strain 186
ANPGLIIGALAG

SEQ ID NO: 75 - protein, 12 aa, Herpes Simplex Virus 2 Strain 186
NPGLIIGALAGS

SEQ ID NO: 76 - protein, 12 aa, Herpes Simplex Virus 2 Strain 186
PGLIIGALAGST